(19) United States
(12) Patent Application Publication
(10) Pub. No.: US 2017/0313021 A1
(43) Pub. Date: Nov. 2, 2017

(54) INFLATABLE MEDICAL ARTICLES

(71) Applicant: 3M INNOVATIVE PROPERTIES COMPANY, ST. PAUL, MN (US)

(72) Inventors: JAYANT CHAKRAVARTY, WOODBURY, MN (US); MATTHEW T. SCHOLZ, WOODBURY, MN (US); HAOMING RONG, WOODBURY, MN (US); KOREY W. KARLS, WOODBURY, MN (US)

(73) Assignee: 3M INNOVATIVE PROPERTIES COMPANY, St. Paul, MN (US)

(21) Appl. No.: 15/520,091

(22) PCT Filed: Oct. 27, 2015
(86) PCT No.: PCT/US2015/057501

§ 371 Date: Apr. 19, 2017

Related U.S. Application Data
(60) Provisional application No. 62/069,934, filed on Oct. 29, 2014.

(51) Int. Cl. B32B 3/28; A61F 7/08; A61L 31/04; A61L 31/06; A61L 31/14; B32B 5/02; B32B 5/26; A61F 7/02; B32B 7/12; B32B 27/36; B32B 27/32; B32B 27/12; B32B 27/08; B32B 7/04; A61F 7/00

(52) U.S. Cl. CPC B32B 3/28; B32B 7/045; A61F 7/08; A61L 31/048; A61L 31/06; A61L 31/14; B32B 5/022; B32B 5/26; A61F 7/02; B32B 7/12; B32B 27/36; B32B 27/32; B32B 27/12; B32B 27/08; A61F 2007/0244; A61F 2007/006; A61F 2007/0091; A61F 2007/0233; B32B 2437/00; B32B 2262/0276; B32B 2307/7242; B32B 2307/7265; B32B 2262/0253

(57) ABSTRACT

An inflatable medical article that includes: a polyester-containing layer including a fabric layer that includes at least one nonwoven web of fibers including an aliphatic polyester, wherein at least a portion of the aliphatic polyester is exposed at the surface of the fibers; a polyolefin-containing layer including a polyolefin film, a nonwoven web including polyolefin fibers, or a combination thereof; a tie layer bonding the polyester-containing layer to the polyolefin-containing layer; an optional sheet; and at least one inflatable chamber formed between the polyolefin-containing layer and the tie layer, or between the polyester-containing layer and the tie layer, or between the polyolefin-containing layer and the optional sheet (when the sheet is present).

### CROSS REFERENCE TO RELATED APPLICATIONS

This application claims priority to U.S. Provisional Patent Application No. 62/069,934, filed Oct. 29, 2014, the disclosure of which is incorporated by reference in its entirety herein.

### BACKGROUND

A patient warming device, particularly a disposable warming device can be in the form, for example, of a blanket, pad, or a garment. For example, a clinical blanket or garment can be used to temporarily clothe a patient or clinician in a clinical setting. Such blankets and garments include hospital blankets, gowns, robes, bibs, and other equivalent articles. The clinical setting may be a medical or dental office or clinic, a hospital, or any facility or institution that provides medical or dental treatment to patients. In some cases, a warming device includes at least one convective apparatus attached to or integrated with the device, e.g., blanket or garment. A convective apparatus receives and distributes at least one stream of inflating medium in a structure for being disposed on, adjacent to, or next to the core and/or the limbs of a body. When pressurized with warmed air, a convective apparatus emits warmed air through one or more of its surfaces. The emission of inflating medium can be through mechanical openings for example, holes, apertures, interstices, slits, and the like; or using air permeable materials. Thus, such warming devices are examples of medical inflatable medical articles.

Traditionally, such inflatable medical articles (e.g., warming blankets, pads, and warming or cooling surgical garments) include materials made from petroleum-based thermoplastic polymers such as polyolefins. There is a growing interest in replacing these petroleum-based polymers with resource renewable polymers, i.e., polymers derived from plant based materials. Ideal resource renewable polymers are “carbon dioxide neutral” meaning that as much carbon dioxide is consumed in growing the plant-based material as is given off when the product is made and disposed of. Thus, there is a desire for medical inflatable medical articles that include a greater amount of resource renewable polymers and less petroleum-based polymers.

### SUMMARY

The present disclosure relates to inflatable medical articles.

In one embodiment, the inflatable medical article includes: a polyester-containing layer including a fabric layer that includes at least one nonwoven web of fibers including an aliphatic polyester, wherein at least a portion of the aliphatic polyester is exposed at the surface of the fibers; a polyolefin-containing layer including a polyolefin film, a nonwoven web including polyolefin fibers, or a combination thereof; a tie layer bonding the polyester-containing layer to the polyolefin-containing layer; an optional sheet; and at least one inflatable chamber formed between the polyolefin-containing layer and the tie layer, or between the polyester-containing layer and the tie layer, or between the polyolefin-containing layer and the optional sheet (when the sheet is present). The inflatable medical article may further include a plurality of engineered openings for fluid communication between the at least one inflatable chamber and the environment.

The polyester-containing layer is in the form of a fabric layer that includes at least one nonwoven web of fibers including an aliphatic polyester, wherein at least a portion of the aliphatic polyester is exposed at the surface of the fibers. In certain embodiments, the aliphatic polyester is selected from the group of poly(lactide), poly(glycolide), poly(lactide-co-glycolide), poly(L-lactide-co-trimethylene carbonate), poly(dioxanone), poly(butylene succinate), poly(butylene adipate), poly(ethylene adipate), polyhydroxybutyrate, polyhydroxyvalerate, and blends and copolymers thereof. Preferably, the selected aliphatic polyesters are obtained from renewable resources, such as poly(lactic acid).

The polyolefin-containing layer may be in the form of a fabric layer or a film layer, or a combination thereof. The polyolefin-containing layer has at least a portion of the polyolefin exposed at a surface of the layer that is adjacent to a tie layer that bonds the polyolefin-containing layer to the polyester-containing layer. In some embodiments, the polyolefin includes at least one of polyethylene and polypropylene. In some embodiments, the polyethylene includes at least one of low density polyethylene and linear low density polyethylene.

The tie layer includes a copolymer prepared from monomers including at least one olefin monomer and at least one polar monomer (e.g., up to 22 wt-% of at least one polar monomer). In some embodiments, the copolymer of the tie layer has a Vicat softening temperature of greater than 45° C. In some embodiments, the entire tie layer composition has a Vicat softening temperature of greater than 45° C.

In some embodiments, the copolymer of the tie layer further includes at least one reactive monomer, wherein the reactive monomer includes a reactive group that is capable of reacting with and covalently bonding to a hydroxyl group (e.g., at elevated temperatures that can be reached during extrusion). In some embodiments, the tie layer further includes a reactive polymer having at least one reactive monomer, wherein the reactive monomer includes a reactive group that is capable of reacting with and covalently bonding to a hydroxyl group. In some embodiments, the tie layer further includes a tackifier.

In some embodiments of the tie layer, at least one olefin monomer is ethylene. In some embodiments, at least one polar monomer is selected from the group consisting of vinyl acetate, a (C1-C8)alkyl ester of (meth)acrylic acid, a (C1-C4)acrylic acid, and combinations thereof. In some embodiments, the reactive group of the reactive monomer is an anhydride group or an epoxy group. In some embodiments, the tie layer includes a thermoplastic elastomer. In some embodiments, the thermoplastic elastomer is a block copolymer including alkyl methacrylate and alkyl acrylate blocks, e.g., a poly(methyl methacrylate)-poly(butyl acrylate)-poly(methyl methacrylate) copolymer. In some embodiments, the tie layer further includes an alkyl benzoate plasticizer.

The present disclosure also provides a medical device that includes an inflatable medical article as described herein and a convective apparatus integrated with or attached to the inflatable medical article.

The terms “polymer” and “polymeric material” (including elastomer and elastomeric polymer) include, but are not limited to, organic homopolymers, copolymers, and the like, such as for example, block, graft, random and alternating copolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries, as well as linear, branched, hyperbranched, and dendritic forms. Herein, “copolymer” is used to encompass organic polymers including two or more different monomers (including copolymers, terpolymers, tetrapolymers, etc.).

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The words “preferred” and “preferably” refer to claims of the disclosure that may afford certain benefits, under certain circumstances. However, other claims may also be preferred, under the same or other circumstances. Furthermore, the recitation of one or more preferred claims does not imply that other claims are not useful, and is not intended to exclude other claims from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.). Herein, “up to” a number (e.g., up to 50) includes the number (e.g., 50).

As used herein, the term “room temperature” refers to a temperature of about 20° C. to about 25° C. or about 22° C. to about 25° C.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the invention. Thus, the appearances of such phrases in various places throughout this specification are not necessarily referring to the same embodiment of the invention. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIGS. 1A and 1B are cross-sections exemplary laminates that include at least one polyester-containing layer, at least one polyolefin-containing layer, and at least one tie layer, according to some embodiments of the present disclosure.

FIG. 2A is a cross-section of a portion of an inflatable medical article that includes a laminate of FIG. 1A bonded to a sheet at discrete locations thereby forming inflatable chambers.

FIG. 2B and FIG. 2C are cross-sections of portions of alternative inflatable medical articles that include a laminate of FIG. 1A, wherein the layers are bonded at discrete locations thereby forming inflatable chambers within the laminate.

FIG. 3A and FIG. 3B are cross-sections of portions of alternative inflatable medical articles according to some embodiments of the present disclosure.

FIG. 4 is a cross-section of a portion of an inflatable medical article that includes a laminate bonded to a sheet, wherein the sheet is another laminate.

FIG. 5A is a schematic of a common upper-body patient warming product, with a cross-section showing inflatable (i.e., uninflated) chambers in FIG. 5B, and a cross-section showing inflated chambers in FIG. 5C.

### DETAILED DESCRIPTION OF ILLUSTRATIVE EMBODIMENTS

The present disclosure relates to inflatable medical articles. Generally, such articles include a laminate that includes at least one polyester-containing layer, at least one polyolefin-containing layer, and at least one tie layer bonding the polyester-containing layer to the polyolefin-containing layer. In some embodiments, a laminate (which may be a co-extruded construction of the three layers) is bonded to a sheet at discrete locations thereby forming one or more inflatable chambers. In some embodiments, the layers within the laminate are bonded at discrete locations thereby forming one or more inflatable chambers within the laminate.

More specifically, an inflatable medical article of the present disclosure includes: a polyester-containing layer including a fabric layer that includes at least one nonwoven web of fibers including an aliphatic polyester, wherein at least a portion of the aliphatic polyester is exposed at the surface of the fibers; a polyolefin-containing layer including a polyolefin film, a nonwoven web including polyolefin fibers, or a combination thereof; a tie layer bonding the polyester-containing layer to the polyolefin-containing layer; an optional sheet; and at least one inflatable chamber formed between the polyolefin-containing layer and the tie layer, or between the polyester-containing layer and the tie layer, or between the polyolefin-containing layer and the optional sheet when present. The inflatable medical article may further include a plurality of engineered openings for fluid communication between the at least one inflatable chamber and the environment. Such openings may be useful for the emission of the inflating medium (e.g., warm air).

Such inflatable medical articles can be used as warming devices in the form of, for example, a blanket, pad, or garment (e.g., a disposable garment). For example, a blanket or garment can be used in a clinical setting to temporarily clothe, cover, or support a patient or clinician, and include hospital blankets, gowns, robes, bibs, and other equivalent articles. Warming blankets and pads may be placed on top of, beneath, or surrounding the patient. Clinical garments, for example, can be used for warming.

The clinical setting may be a medical or dental office or clinic, a hospital, or any facility or institution that provides medical or dental treatment to patients. In some cases, an inflatable medical article (e.g., in the form of a blanket, pad, or garment) can be a warming device with at least one convective apparatus attached to or integrated with the inflatable medical article.

A convective apparatus receives and distributes at least one stream of inflating medium in a structure for being disposed on, adjacent to, or next to the core and/or the limbs of a body. When pressurized with warmed air, a convective apparatus emits warmed air through one or more of its surfaces. The emission of inflating medium can be through engineered openings for example, holes, apertures, interstices, slits, and the like, or through the use of air permeable materials. The resultant inflatable medical articles are useful as warming devices.

For example, such an inflatable medical article may be worn on a person where it receives a stream of warmed pressurized air, distributes the pressurized air within a convective apparatus, and emits the air through one or more surfaces of the convective apparatus to warm the person's body. Such articles are flaccid when not in use and become taut when receiving a stream of pressurized air.

Exemplary medical device (e.g., warming device) constructions include an inflatable medical article (e.g., a clinical garment) and one or more convective apparatuses integrated with or attached to the inflatable medical article. The convective apparatus is typically attached to the inflatable medical article such that the inflating medium passes through inlet ports. In some aspects, an elongated upper body convective apparatus in the upper portion of the clinical garment extends between the sleeves. In some aspects, a lower, multi-section convective apparatus is integrated with or attached to the clinical garment, beneath the upper portion. In yet other aspects, both upper body and lower multi-section convective apparatuses are integrated with or attached to the clinical garment, from the upper portion to the lower hem.

Additionally exemplary medical device (e.g., warming device) constructions include a convective warming blanket or pad that can be placed on the patient or under the patient. An example of an over-body blanket is an upper body blanket that is placed on the patient to cover the upper torso, head, and arms. Once in place, warmed air inflates the blanket and is emitted onto the body. An example of an under-body blanket or pad is an inflatable convective pad that is placed under a patient. Once the patient is placed on the pad, warmed air inflates the pad and bathes the patient with warm air.

Inflatable medical articles of the present disclosure are preferably operated with air at a temperature greater than 38° C. (or greater than 43° C., and often up to 48° C.), measured at the blower/warming unit hose exit, such as that of 3M BAIR HUGGER 500 or 700 series warming units. It may be useful to operate such inflatable medical articles with pressurized air that includes a mixture of selected constituents including water vapor, medicaments, scented compounds, and the like.

Inflatable medical articles of the present disclosure include a polyester-containing layer, a polyolefin-containing layer, and a tie layer bonding the polyester-containing layer to the polyolefin-containing layer. The polyester-containing layer is typically in the form of a fabric layer. The polyolefin-containing layer can be in the form of a film layer or a fabric layer. Multiple layers of polyester-containing, polyolefin-containing, and tie layers may be used. In one aspect of this disclosure, the tie layers provide suitable bonding for incompatible polyester-containing layers to polyolefin-containing layers, wherein the bond withstands high pressures and temperatures.

Various configurations can be created using a polyester-containing layer, polyolefin-containing layer, tie layer, and an optional sheet to form at least one inflatable chamber. For example, one or more inflatable chambers may be formed between the polyolefin-containing layer and the tie layer, or between the polyester-containing layer and the tie layer, or between the polyolefin-containing layer and the optional sheet when present. In the latter configuration, the polyester-containing layer, the polyolefin-containing layer, and the tie layer form a laminate, and the sheet is bonded to the laminate to create at least one inflatable chamber.

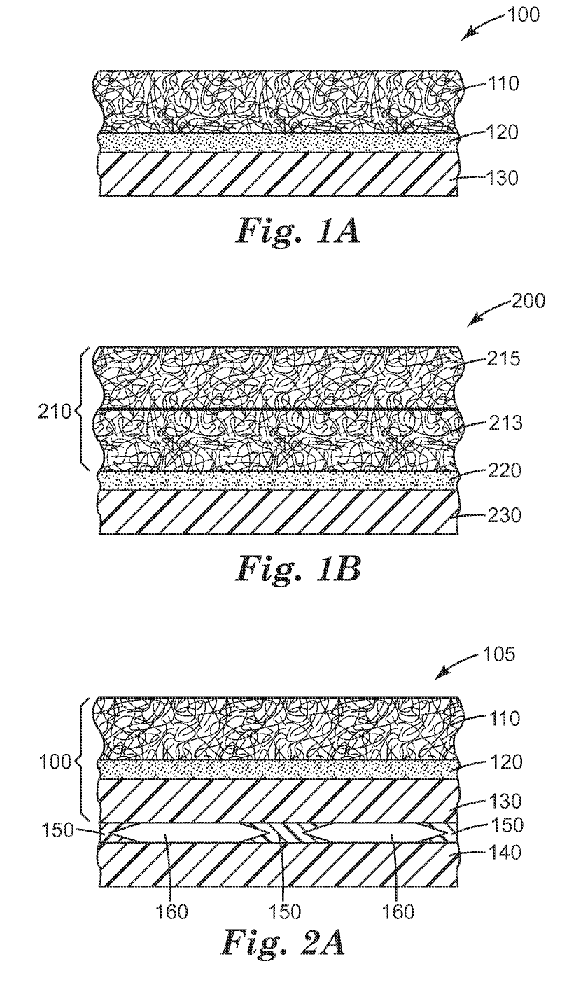

Referring to FIG. 1A, in some embodiments, a polyester-containing layer in the form of a fabric layer 110 of laminate 100 is bonded to a polyolefin-containing layer in the form of a film layer 130 using tie layer 120. The fabric layer 110 includes at least one nonwoven web of fibers that include an aliphatic polyester, wherein at least a portion of the aliphatic polyester is exposed at the surface of the fibers. The polyolefin-containing film layer 130 includes a polyolefin, at least a portion of which is exposed at the surface adjacent to tie layer 120. Although each layer is shown as a single ply (and not necessarily to scale), in some embodiments, two or more plies may be used. For example, the fabric layer 110 can be in the form of two or more plies, i.e., two or more nonwoven webs of fibers, which may be the same or different. In certain embodiments, the polyester-containing layer may be in the form of a film, or a combination of two or more layers of nonwoven webs of fibers and films.

Referring to FIG. 1B, laminate 200 includes a polyester-containing fabric layer 210 and a polyolefin-containing film layer 230. Polyester-containing fabric layer 210 includes at least two plies. For example, first ply 213 may include different additives than the second ply 215. The first ply 213 is indirectly bonded to polyolefin-containing film layer 230 using a tie layer 220. Polyolefin-containing film layer 230 includes a polyolefin, at least a portion of which is exposed at the surface adjacent to tie layer 220. In some embodiments, first ply 213 may be a polyester-containing film compatible with the polyester-containing fabric layer 210.

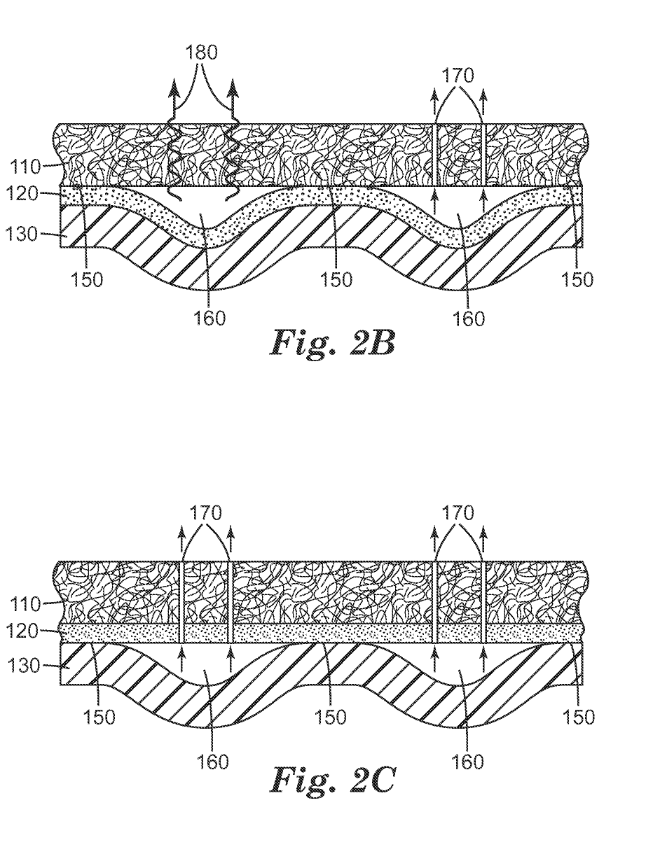

Such laminates (100 or 200 from FIG. 1A or 1B) can be used with a sheet bonded thereto to create at least one inflatable chamber of an inflatable medical article. The sheet can be in the form of a film (i.e., film layer) or another laminate. For example, as shown in FIG. 2A, a cross-section of a portion of an inflatable medical article 105 is shown that includes a laminate 100 that includes a polyester-containing fabric layer 110, a tie layer 120, and a polyolefin-containing film layer 130. The inflatable medical article 105 also includes a sheet 140 bonded to the laminate 100 at discrete bonding points 150 to create at least one inflatable chamber 160. In this embodiment, sheet 140 is a film layer, which may or may not be of the same material of the polyolefin-containing film layer 130 of the laminate 100. Polyolefin-containing film layer 130 includes a polyolefin, at least a portion of which is exposed at the surface adjacent to tie layer 120. The bonding points 150 may be of the same material as one or both of the film layers 130 and 140, or may be a different than both film layers (e.g., an adhesive).

The polyolefin-containing film layer 130 and/or the tie layer 120 provides a sufficient fluid (e.g., gas and/or liquid) barrier to allow for the formation of inflatable chambers. As used herein the term “barrier” refers to a material for making the layer that does not allow air to pass through the material per se but directs the air through the engineered openings.

Although FIG. 2A demonstrates a configuration in which one or more inflatable chambers are formed between the polyolefin-containing layer and a sheet, other configurations are possible that do not include the sheet. For example, configurations in which one or more inflatable chambers are formed within the laminate of the polyester-containing layer, polyolefin-containing layer, and tie layer are possible. That is, configurations can include one or more inflatable chambers formed between the polyester-containing layer and the tie layer, or between the polyolefin-containing layer and the tie layer, as shown in FIGS. 2B and 2C, respectively.

Referring to FIG. 2B, in some embodiments, a polyester-containing layer in the form of a fabric layer 110 is bonded at discrete bonding points 150 to a tie layer 120 that is laminated to a polyolefin-containing layer in the form of a film layer 130, thereby forming at least one inflatable chamber 160. Referring to FIG. 2C, in some embodiments, a polyester-containing layer in the form of a fabric layer 110 is laminated to a tie layer 120 that is bonded at discrete bonding points 150 to a polyolefin-containing layer in the form of a film layer 130, thereby forming at least one inflatable chamber 160. Polyolefin-containing film layer 130 includes a polyolefin, at least a portion of which is exposed at the surface adjacent to tie layer 120. The fabric layer 110 (of either FIG. 2B or 2C) includes at least one nonwoven web of fibers that include an aliphatic polyester, wherein at least a portion of the aliphatic polyester is exposed at the surface of the fibers.

This polyester-containing fabric layer 110 of the embodiments shown in FIGS. 2A and 2B can include multiple plies, which can provide sufficient fluid/gas barrier to allow for the formation of inflatable chambers. Alternatively, the combination of polyester-containing fabric layer and the tie layer provide a fluid barrier to allow for the formation of inflatable chambers. For example, in the embodiment shown in FIG. 2C, where inflatable chambers 160 are formed between the tie layer 120 and the film layer 130, the tie layer 120 may be coextruded onto the polyester-containing fabric layer 110 and then subsequently the polyolefin-containing layer may be point bonded to the coextruded tie layer/polyester-containing fabric layer.

It should be noted that no layers in any of the exemplary embodiments of the figures shown herein are to scale. Furthermore, although chambers such as the chambers 160 in FIGS. 2A, 2B, and 2C are described as inflatable, they are shown in a slightly inflated form because when completely uninflated, a cross-section would not show the chambers. Such embodiments could be used to form a variety of inflatable medical articles, but is particularly suitable for use in making an over-body or under-body warming blanket (i.e., thermal blanket) with the side that contacts the patient being the fabric layer 110.

The inflatable medical articles may further include a plurality of engineered openings for fluid communication between the at least one inflatable chamber and the environment. These openings 170 are shown in FIGS. 2B and 2C. These openings 170 may be in the form of slits, holes, or the like. It should be understood, however, that such engineerd openings 170 may not be necessary, as sufficient air permeability (as shown in FIG. 2B along lines 180) may be provided by the predetermined natural porosity of the polyester-containing fabric layer 110.

As used herein, an “engineered” opening shall mean a structure deliberately formed into and integral with a surface. These openings are typically in a defined pattern. An engineered structure may be created, for example, by forming or perforating holes, apertures, interstices, slits, and the like, in a specific pattern unto a surface. Such engineered mechanical openings for example, holes, apertures, interstices, slits, and the like are integrated with the inflatable chambers. By “engineered” it is meant that the size and shape of the mechanical openings or the size and shape of the openings in the air permeable materials are not random, but are predetermined and fabricated in such a way so that the inflatable chamber will stay inflated given the appropriate level of constant pressure of supplied warm air, while at the same time allow a controlled flow of that air out of the inflatable chambers of the inflatable device towards via the engineered mechanical openings or via the engineered air permeable materials in order to warm the patient.

An air permeable material may also be in the form of a nonwoven which has been flat bonded by going through smooth thermal calender rolls with sufficient heat and pressure so that the nonwoven fibers are compressed together to form microporous structures between the fibers with low air permeability. The flat bonded, calendered nonwoven web may be further perforated or slit to form holes, apertures, interstices, slits, so as to provide the desired flow of warm air over the patient. Alternatively, the calendered nonwoven web may be compressed so that the microporous structures themselves formed between the fibers are engineered to provide the desired controlled flow of warm air over the patient.

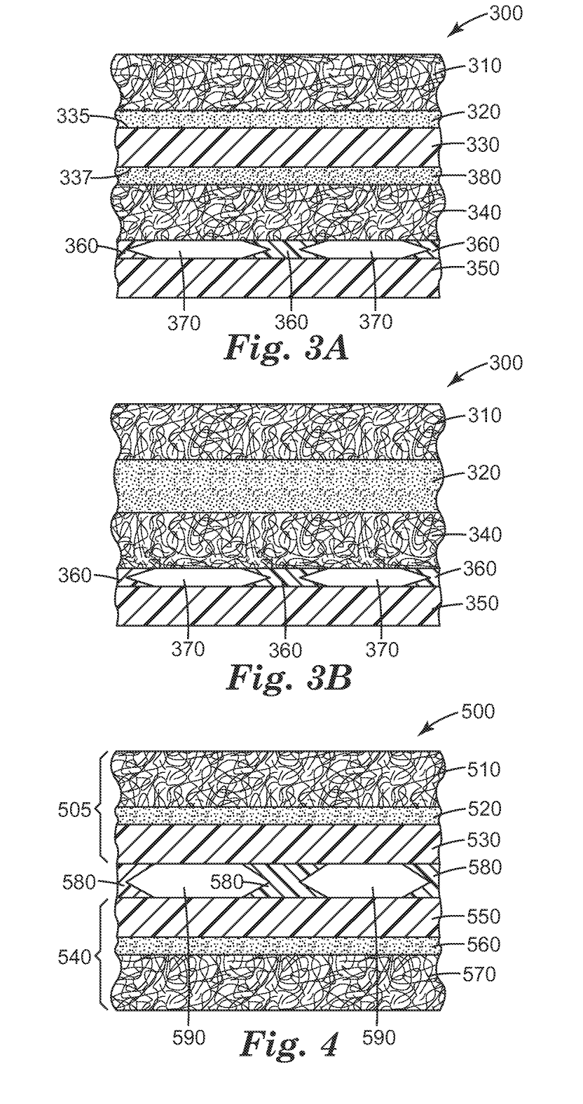

FIG. 3A and FIG. 3B show cross-sections of portions of alternative inflatable medical articles. Referring to FIG. 3A, a cross-section of a portion of an inflatable article 300 is shown. In this exemplary embodiment, a first fabric layer 310 is bonded to a first major surface 335 of a polyolefin-containing film layer 330 using a first tie layer 320, and a second fabric layer 340 is bonded to a second major surface 337 of the polyolefin-containing film layer 330 using a second tie layer 380. At least one of the fabric layers 310 and 340 is a polyester-containing layer that includes at least one nonwoven web of fibers that includes an aliphatic polyester, wherein at least a portion of the aliphatic polyester is exposed at the surface of the fibers. The other fabric layer can be a polyester-containing fabric layer or a polylefin-containing fabric layer (i.e., one that includes at least one nonwoven web of fibers that includes a polyolefin). In some embodiments, the fabric layer 310 is a polyester-containing fabric layer, and the fabric layer 340 is a polyolefin-containing fabric layer. If the fabric layer 340 is a polyolefin-containing fabric layer, a second tie layer 380 may not be needed because it could be directly bonded to the polyolefin-containing film layer 330. Although the polyolefin-containing layer 330 is shown as a film layer, it can be in the form of a fabric layer that includes a nonwoven web of fibers that include a polyolefin. Polyolefin-containing film layer 330 includes a polyolefin, at least a portion of which is exposed at the surfaces adjacent to tie layers 320 and 380. Layers 310, 320, 330, 340, and 380 in FIG. 3A, and layers 310, 320, and 340 in FIG. 3B form an exemplary laminate of the present disclosure.

Referring to FIG. 3B, a cross-section of a portion of an alternative embodiment of an inflatable article 300 is shown, wherein the polyolefin-containing film layer 330 and the second tie layer 380 of the laminate shown in FIG. 3A are removed. In this embodiment, a first fabric layer 310 is bonded to a second fabric layer 340 using a tie layer 320. At least one of fabric layers 310 and 340 includes at least one nonwoven web of fibers that include an aliphatic polyester, wherein at least a portion of the aliphatic polyester is exposed at the surface of the fibers. Typically, the fabric layer 310 is a polyester-containing fabric layer, and the fabric layer 340 is a polyolefin-containing fabric layer. In this embodiment, tie layer 320 of FIG. 3B serves the purpose of the film layer 330 of FIG. 3A by forming a fluid barrier, typically a gas barrier.

Each of the embodiments shown in FIG. 3A and FIG. 3B shows a sheet 350 bonded to the fabric layer 340 of the laminate at bonding points 360 to create at least one inflatable chamber 370. In this embodiment, sheet 350 is a film layer, which may or may not be of the same material of fabric layer 340. The bonding points 360 may be of the same material as one or both of the layers 340 and 350, or may be a different than both layers (e.g., an adhesive). Also, in this embodiment, two inflatable chambers 370 are shown. Such embodiment could be used to form a variety of inflatable medical articles, but is particularly suitable for use in making an over-body warming blanket (i.e., thermal blanket) with the side that contacts the patient being the polyester-containing fabric layer 310.

Although each layer of FIG. 3A and FIG. 3B is shown as a single ply (and not necessarily to scale), in some embodiments, two or more plies may be used. For example, the fabric layers 310 and/or 340 can be in the form of two or more nonwoven webs of fibers, which may be the same or different. The film layer 330 can also be in form of two or more plies.

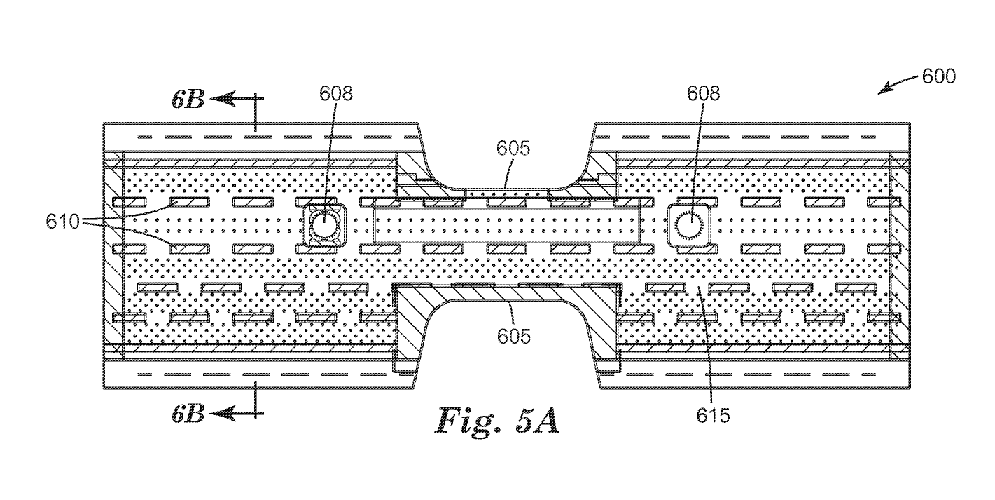

As an alternative embodiment, as shown in FIG. 4, a cross-section of a portion of an inflatable medical article 500 is shown that includes a first laminate 505 that includes a fabric layer 510, a tie layer 520, and a film layer 530. The inflatable medical article 500 also includes a sheet 540, which is in the form of a second laminate. This sheet or second laminate 540 includes a film layer 550, a tie layer 560, and a fabric layer 570, each of which may be the same or different than the fabric layer, tie layer, and film layer of the first laminate 505. This sheet or second laminate 540 is bonded to the first laminate 505 at bonding points 580 to create at least one inflatable chamber 590. The bonding points 580 may be of the same material as one or both of the film layers 530 and 550, or may be a different than both film layers (e.g., an adhesive). In this embodiment, two inflatable chambers 590 are shown. Such embodiment could be used to form a variety of inflatable medical articles, but is particularly suitable for use in making an under-body warming blanket (i.e., thermal blanket).

It should be understood that if all the inflatable space within an inflatable medical article of the present disclosure is connected such that it all inflates from a single inlet, one chamber exists.

The bonding between the laminate and sheet to create at least one inflatable chamber can be accomplished through a variety of well-known techniques. Such techniques include, for example, the application of heat and pressure, the use of ultrasonic bonding, the use of an adhesive, radio-frequency (RF) welding, and the like.

Such inflatable chambers can be in a variety of shapes and sizes. They can be in the form of a plurality of discreet inflatable chambers or one continuous inflatable chamber throughout an inflatable medical article. The chamber(s) may be inflated by pressurized air, typically pressurized heated air, from one or more air sources through one or more inlets. Discreet chambers may be completely separate from each other. In such embodiments, separate sources of pressurized air may be used. Discreet chambers may also be defined as discreet areas of inflated chambers fluidly interconnected but distinguished from each other by differently sized and shaped bonding points that may define the discreet chambers and the periphery of the chambers. Typically, however, if all the inflatable space within an inflatable medical article is connected such that it inflates from a single inlet, one chamber exists.

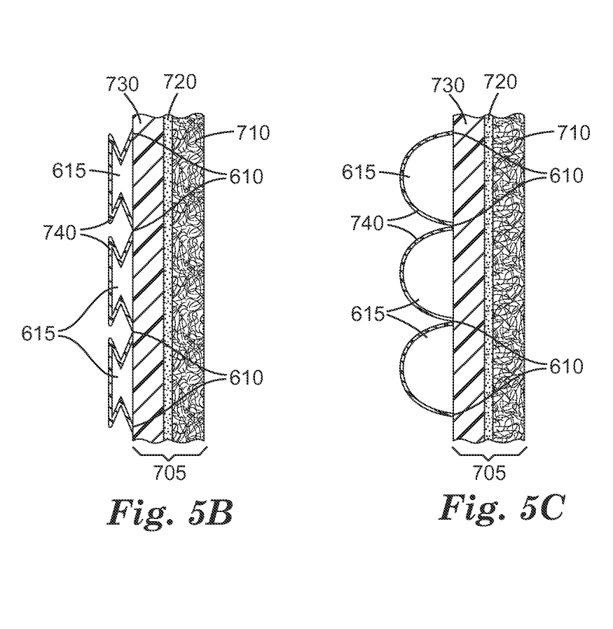

For example, a schematic of a common upper-body patient warming product 600 is shown in FIG. 5A. The product 600 would typically cover a patient's outstretched arms with one of the cutout portions 605 being placed at the patient's neck. The bonding points 610 may be in a regular or random pattern. In this illustration, the bonding points 610 are rectangular and result in one continuous inflatable chamber 615. An uninflated (i.e., inflatable) cross-section of the upper-body patient warming product 600 is shown in FIG. 5B and an inflated cross-section is shown in FIG. 5C, each of which shows the bonding points 610 and the chambers 615, uninflated (i.e., inflatable) (in FIG. 5B) and inflated (in FIG. 5C). FIG. 5A also shows inlet ports 608 through which an inflating medium (e.g., warm air) passes into the chambers 615.

In FIG. 5A, the product 600 is made of a construction similar to that shown in FIG. 2A. This is more clearly seen in FIG. 5B and FIG. 5C, wherein the laminate 705 includes a polyester-containing fabric layer 710, a tie layer 720, and a polyolefin-containing film layer 730, which is bonded to a sheet 740 at bonding points 610 to create at least one inflatable chamber 615. In this embodiment, sheet 740 is a film layer, which may or may not be of the same material of the polyolefin-containing film layer 730 of the laminate 705. The sheet 740 is folded to form pleats when the product is in its uninflated state. In an alternative embodiment, FIG. 5B and FIG. 5C illustrate an inflatable medical article wherein the laminate 705 includes a polyester-containing fabric layer 710, a tie layer 720, a polyolefin-containing film layer 730, and a sheet 740 (not drawn to scale), which forms inflatable chamber (s) 615 between polyolefin-containing film layer 730 and sheet 740.

Typically, an inflatable medical article of the present disclosure (e.g., a warming device) is soft, flexible, and drape over/on an individual (e.g., a patient) when uninflated and relatively quiet when deployed over the patient or crumpled. Aliphatic polyesters are often high in modulus. Films made from aliphatic polyesters such as polylactic acid are typically quite stiff and very noisy when crumpled; however, since the fibers of the fabric are small (generally having a diameter of less than 20 microns and preferably less than 16 microns) the aliphatic polyester fabric is soft, flexible, and drapeable. By contrast, polyolefin fabrics and films are very flexible and quiet.

It has proven to be very difficult to bond incompatible aliphatic polyester fiber-containing fabric layers with polyolefin-containing fabric or film layers described herein, wherein the bonds do not fail during use, particularly under elevated temperature and pressure (warm air inflating the device). Patient warming products require high-temperature resiliency in the laminates to withstand hot air flow through the tubes. The hot air makes the tie layer material of the film softer, which results in delamination of the polyolefin-containing film/fabric (i.e., film or fabric) from the polyester-containing fabric. This results in the seals failing constituting a serious product failure. Significantly, the tie layers described herein are suitable for bonding the polyester-containing fabric layers to the incompatible polyolefin film/fabric layers. Accordingly, the tie layers described herein allow the bonding points to withstand high pressures and temperatures in use.

For example, in certain embodiments, inflatable medical articles of the present disclosure can withstand inflation at a pressure of at least 0.1 inch water (2.5 mm water), or at least 0.5 inch water (12.5 mm water), or at least 1.0 inch water (25 mm water), or at least 1.5 inches water (37.5 mm water), or at least 2.0 inches water (50 mm water) in the inflatable medical article (e.g., blanket) with air at a temperature greater than 38° C. (or greater than 43° C., and often up to 48° C.), measured at the blower exit/air inlet of a convective apparatus (i.e., inflating device) according to the Pressure Seal Testing described in the Examples Section, without failure at the tie layer by separation of the polyolefin-containing layer or the polyester-containing layer, or both from the tie layer.

In certain embodiments, inflatable medical articles of the present disclosure can withstand inflation at a pressure of up to 10 inches water (250 mm water), or up to 3.5 inches water (87.5 mm water) in the inflatable medical article (e.g., blanket) with air at a temperature greater than 38° C. (or greater than 43° C., and often up to 48° C.), measured at the blower exit of a convective apparatus (i.e., inflating device) according to the Pressure Seal Testing described in the Examples Section, without failure at the tie layer by separation of the fabric layer from the film layer.

The laminates and bonded sheets described herein can be used in making a wide-variety of inflatable medical articles. For example, they can be used in making warming (i.e., thermal) blankets, thermal or convective pads, gowns, and the like, as described, for example, in U.S. Pat. No. 5,674,269 (Augustine), U.S. Pat. No. 5,697,963 (Augustine), U.S. Pat. No. 5,928,274 (Augustine), U.S. Pat. No. 6,102,936 (Augustine et al.), U.S. Pat. No. 6,176,870 (Augustine), U.S. Pat. No. 7,837,721 (Augustine et al.), U.S. Pat. No. 7,819,911 (Anderson et al.), U.S. Pat. No. 7,846,192 (Panser et al.), U.S. Pat. No. 8,105,370 (Augustine), U.S. Pat. No. 8,177,828 (Anderson et al.), and U.S. Pat. No. 8,460,354 (Anderson et al.) or PCT Publication No. WO 2015/095129 (McGregor).

### Polyester-Containing Fabric Layer

The polyester-containing fabric layer includes at least one nonwoven web of fibers that include an aliphatic polyester, wherein at least a portion of the aliphatic polyester is exposed at the surface of the fibers (and, hence, at the surface of the nonwoven web). The polyester-containing fabric layer may include two or more nonwoven webs that include an aliphatic polyester, wherein at least a portion of the aliphatic polyester is exposed at the surface of the fibers (and, hence, at the surface of each nonwoven web).

In certain embodiments, the polyester-containing fabric layer may include at least one nonwoven web of polyester-containing fibers and at least one polyester film or polyester-compatible film laminated to the at least one nonwoven web of polyester-containing fibers.

In certain embodiments, at least 50% of the exterior surface area of the fibers and/or at least 50% of the surface area of the nonwoven web includes an aliphatic polyester. In certain embodiments, at least 75% of the exterior surface area of the fibers and/or at least 75% of the surface area of the nonwoven includes an aliphatic polyester.

In some embodiments, nonwoven webs are formed from multicomponent fibers such as bicomponent fibers. In some embodiments it may be desirable to use a sheath/core or side by side fiber construction where the sheath or one side includes the aliphatic polyester, such that at least a portion of the aliphatic polyester is exposed at the surface of the fibers. In a particularly preferred embodiment the fibers are spunbond fibers formed using a bicomponent sheath/core die where the sheath includes the aliphatic polyester.

Exemplary aliphatic polyester include poly(lactide) (also known as poly(lactic acid) or PLA), poly(glycolide), poly(lactide-co-glycolide), poly(L-lactide-co-trimethylene carbonate), poly(dioxanone), poly(ethylene succinate), poly(butylene succinate), poly(butylene adipate), poly(ethylene adipate), polyhydroxybutyrate, polyhydroxyvalerate, and blends and copolymers thereof.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

Poly(lactide)s may be prepared as described in U.S. Pat. No. 6,111,060 (Gruber, et al.), U.S. Pat. No. 5,997,568 (Liu), U.S. Pat. No. 4,744,365 (Kaplan et al.), U.S. Pat. No. 5,475,063 (Kaplan et al.), U.S. Pat. No. 6,143,863 (Gruber et al.), U.S. Pat. No. 6,093,792 (Gross et al.), U.S. Pat. No. 6,075,118 (Wang et al.), U.S. Pat. No. 5,952,433 (Wang et al.), U.S. Pat. No. 6,117,928 (Hiltunen et al.), and U.S. Pat. No. 5,883,199 (McCarthy et al.), as well as WO 98/24951 (Tsai et al.), WO 00/012606 (Tsai et al.), WO 84/04311 (Lin), WO 99/50345 (Kolstad et al.), WO 99/006456 (Wang et al.), WO 94/07949 (Gruber et al.), WO 96/22330 (Randall et al.), and WO 98/50611 (Ryan et al.). Reference may also be made to J. W. Leenslag, et al., J. Appl. Polymer Science, vol. 29, pp. 2829-2842 (1984), and H. R. Kricheldorf, Chemosphere, vol. 43, pp. 49-54 (2001).

Particularly useful aliphatic polyesters include those derived from semicrystalline polylactic acid. Poly(lactic acid) or polylactide has lactic acid as its principle degradation product, which is commonly found in nature, is non-toxic and is widely used in the food, pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, poly(lactide) polymers may be obtained having different stereochemistries and different physical properties, including crystallinity. The L,L- or D,D-lactide yields semicrystalline poly(lactide), while the poly(lactide) derived from the D,L-lactide is amorphous.

The polylactide preferably has a high enantiomeric ratio to maximize the intrinsic crystallinity of the polymer. The degree of crystallinity of a poly (lactic acid) is based on the regularity of the polymer backbone and the ability to crystallize with other polymer chains. If relatively small amounts of one enantiomer (such as D-) is copolymerized with the opposite enantiomer (such as L-) the polymer chain becomes irregularly shaped, and becomes less crystalline. For these reasons, when crystallinity is favored, it is desirable to have a poly(lactic acid) that is at least 85% of one isomer, at least 90% of one isomer, or at least 95% of one isomer in order to maximize the crystallinity.

In certain embodiments, the most preferred PLA is greater than 97% D isomer. In certain cases it may be desirable to blend a PLA polymer that is very high in D isomer (e.g., greater than 98%) with a PLA polymer that is very high in L isomer (e.g., greater than 98%).

An approximately equimolar blend of D-polylactide and L-polylactide is also useful. This blend forms a unique crystal structure having a higher melting point (−210° C.) than does either the D-(polylactide) and L-(polylactide) alone (−160° C.), and has improved thermal stability, see H. Tsuji et. al., Polymer, vol. 40, pp. 6699-6708 (1999).

Copolymers, including block and random copolymers, of poly(lactic acid) with other aliphatic polyesters may also be used. Useful co-monomers include glycolide, betapropiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxy stearic acid.

Preferred materials include biodegradable materials having adequate properties to permit them to break down when exposed to conditions which lead to composting. Examples of materials thought to be biodegradable include aliphatic polyesters such as poly(lactide), poly(glycolide), poly(caprolactone), poly(lactide-co-glycolide), poly(ethylene succinate), polybutylene succinate), polyhydroxybutyrate, and combinations thereof.

Blends of aliphatic thermoplastic polyesters and blends of one or more aliphatic thermoplastic polyesters with one or more of a variety of other polymers including aromatic polyesters, aliphatic/aromatic copolyesters, cellulose esters, cellulose ethers, thermoplastic starches, ethylene vinyl acetate, polyvinyl alcohol, ethylenevinyl alcohol, and the like may also be used. In blended compositions that include thermoplastic polymers which are not aliphatic polyesters, the aliphatic polyester is typically present at a concentration of greater than 60% by weight of total blend, preferably at least 70%, e.g., at least 75%, at least 80%, at least 85%, at least 90%, and even at least 95% by weight of total blend.

The molecular weight of the polymer should be chosen so that the polymer may be processed as a melt. For polylactide, for example, the molecular weight may be from 10,000 to 1,000,000 daltons, and is preferably from 30,000 to 300,000 daltons. By “melt-processable” it is meant that the aliphatic polyesters are fluid or can be pumped or extruded at the temperatures used to process the articles (e.g., make the fine fibers in the nonwoven webs), and do not degrade or gel at those temperatures to the extent that the physical properties are so poor as to be unusable for the intended application. Thus, many of the materials can be made into nonwovens using melt processes such as spunbond, blown micro fiber, and the like. Certain embodiments also may be injection molded.

For multicomponent fibers, the components other than the aliphatic polyester can include one or more of a variety of other polymers including aromatic polyesters, aliphatic/aromatic copolyesters, cellulose esters, cellulose ethers, thermoplastic starches, ethylene vinyl acetate, polyvinyl alcohol, ethylenevinyl alcohol, polyolefins, and the like may also be used. The multicomponent fibers may also include, for example, two different types of aliphatic polyesters, two different blends that include an aliphatic polyester, or two different compositions that include the same aliphatic polyester(s) and different additives (e.g., antishrink additives, tackifiers, surfactants, plasticizers, etc.). For such multicomponent constructions, the aliphatic polyester is typically present at a concentration of greater than 60% by weight of total fiber content, preferably at least 70%, e.g., at least 75%, at least 80%, at least 85%, at least 90%, at least 95%, and even 100%, by weight of total fiber content.

In some embodiments, the fabric layer includes a plurality of continuous fibers including one or more thermoplastic aliphatic polyesters and a thermoplastic antishrinkage additive in an amount greater than 0 wt-% and no more than 15 wt-% of the fibers. In some embodiments, the fibers exhibit molecular orientation. In some embodiments, the fabric layer has at least one dimension which decreases by no greater than 10% in the plane of the layer when the layer is heated to a temperature above a glass transition temperature of the fibers, but below the melting point of the fibers. In some exemplary embodiments, the molecular orientation of the fibers results in a bi-refringence value of at least 0.01. In most embodiments, the fibers are microfibers (i.e., fine fibers or filaments of 1 decitex (dtex) or less, or 0.9 denier or less; depending on the polymer density, this could be 15 microns or less) and particularly fine fibers having an average diameter of less than 20 microns and preferably less than 16 microns. In certain cases it may be desirable to include a fraction of the fibers of larger diameter e.g. greater than 20 microns and perhaps greater than 30 microns. These larger fibers generally would make up less than 20% of the web by weight.

A polyester-containing fabric layer has a basis weight that may be varied depending upon the particular end use. In some embodiments, a polyester-containing fabric layer has a basis weight of at least 1.0 gsm, or at least 10 gsm, or at least 15 gsm. In some embodiments, the fabric layer has a basis weight of up to 1000 grams per square meter (gsm), or up to 500 gsm, or up to 300 gsm, or up to 100 gsm, or up to 60 gsm. In other embodiments, a polyester-containing fabric layer has a basis weight of from 10 gsm to 300 gsm. For use in some applications such as medical fabrics, including disposable warming blankets and gowns, the basis weight is typically from 10 gsm to 100 gsm, e.g., 15 gsm to 60 gsm.

A polyester-containing fabric layer also has a thickness that may be varied depending upon the particular end use. In some embodiments, the fabric layer has a thickness of at least 0.025 mm, or at least 0.25 mm, or at least 0.5 mm, or at least 1.0 mm. In some embodiments, a polyester-containing fabric layer has a thickness of up to 5.0 mm, or up to 3.5 mm, or up to 2.5 mm, or up to 1.0 mm, or up to 0.5 mm, or up to 300 micrometers (μm), or up to 150 μm, or up to 50 μm. Such thicknesses are measured with essentially no pressure applied, using, for example, an optical comparator. In some embodiments, a polyester-containing fabric layer has a thickness of 0.1 mm to 1.0 mm. For use in some applications, such as medical fabrics, including disposable warming blankets and gowns, the thickness is typically 0.1 mm to 1.0 mm, e.g., 0.25 mm to 0.5 mm.

In certain embodiments, a polyester-containing fabric layer is a nonwoven web that is thermally bonded. In one embodiment it is thermally embossed by passing it through a heated calendar with an embossing patterned roll. The embossing increases the tensile strength of the nonwoven. In order to retain softness and drapability the thermally embossed area is generally less than 30% of the projected area and preferably less than 25% of the projected area. Most preferably the embossed area is less than 20% of the projected area.

In certain embodiments, the nonwoven web may be a melt-blown, spun-bond, spun-laced, and/or wet or dry laid (which includes carded and air-laid).

In certain embodiments, a polyester-containing fabric layer includes at least one nonwoven fibrous web, which can be prepared by fiber-forming processes in which filaments of fiber-forming material are formed by extrusion, subjected to orienting forces, and passed through a turbulent field of gaseous currents while at least some of the extruded filaments are in a softened condition and reach their freezing temperature (e.g., the temperature at which the fiber-forming material of the filaments solidifies) while in the turbulent field. Such fiber forming processes include, for example, melt-spinning (i.e., spun-bond), filament extrusion, electrospinning, gas jet fibrillation, or combinations thereof. In some embodiments, the nonwoven fibrous webs can be prepared by fiber-forming processes in which substantially non-molecularly oriented filaments of fiber-forming material are formed using a melt-blowing (e.g., BMF) process.

### Polyester-Containing Fabric Layer Optional Additives

The fibers of the nonwoven webs of the polyester-containing fabric layers may include a variety of optional additives, incorporated into the fibers and/or coated on the fibers. Examples of such optional additives include surfactants, surfactant carriers, antishrink additives, antistatic additives, colorants (pigments and dyes), nucleating agents, antioxidants, plasticizers, and the like. One or more of such additives may be used if desired.

A polyester-containing fabric layer may be inherently fluid repellent or rendered fluid repellent to avoid absorption of blood or other body fluids that may contain pathogenic microorganisms. Qualitatively, “fluid repellent” refers to a material that shows no wicking and no absorption of distilled water at room temperature. For test purposes, a fabric is considered repellent if it does not absorb a 50 μL (microliter) distilled water droplet completely after 60 seconds when placed on the specimen when lying on a flat, smooth, horizontal surface and conditioned for at least 2 hours at 23° C. and 50% relative humidity (RH). For example, a polyester-containing fabric layer may be post-treated with a repellent finish that includes a fluorochemical, silicone, hydrocarbon, or combinations thereof. Exemplary fluorochemicals include a perfluoroalkyl group having at least 4 carbon atoms. These fluorochemicals may be small molecules, oligamers, or polymers. Suitable fluorochemicals may be found in U.S. Pat. No. 6,127,485 (Klun at al.) and U.S. Pat. No. 6,262,180 (Klun et al). Other suitable repellants may include fluorochemicals and silicone fluids repellents disclosed in U.S. Pat. No. 8,721,943 (Moore et al.).

A polyester-containing fabric layer may also optionally include one or more surfactants to help wet the surface and make the fabric absorbent and/or to aid in contacting and killing microorganisms. As used herein, the term “absorbent” means the fabric layer can absorb fluids such as water and aqueous body fluids (e.g., blood) when a droplet is gently placed on the surface. For test purposes, a fabric is considered absorbent if the fabric completely absorbs a 50 μL (microliter) distilled water droplet after 60 seconds when placed on the specimen when lying on a flat, smooth, horizontal surface and conditioned for at least 2 hours at 23° C. and 50% relative humidity. Fabrics are considered highly absorbent if the droplet absorbs in less than 10 seconds. In some embodiments, absorbent fabric layers can absorb over 100% of their weight in water when a single sample is placed on the surface of deionized water for 60 seconds, removed with a tweezers, shaken briskly, and weighed. Preferably, the layer can absorb over 150% of its weight and more preferably over 200% of its weight in water.

As used herein, the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. In applications in which biodegradability is important, it may be desirable to incorporate biodegradable surfactants, which typically include ester and/or amide groups that may be hydrolytic ally or enzymatically cleaved. In certain preferred embodiments in which the fabric layer is absorbent, the surfactants are anionic surfactants selected from the group consisting of alkyl, alkenyl, alkaryl and arakyl sulfonates, sulfates, phosphonates, phosphates and mixtures thereof. Included in these classes are alkylalkoxylated carboxylates, alkyl alkoxylated sulfates, alkylalkoxylated sulfonates, and alkyl alkoxylated phosphates, and mixtures thereof. The preferred alkoxylate is made using ethylene oxide and/or propylene oxide with 0-100 moles of ethylene and propylene oxide per mole of hydrophobe. In certain more preferred embodiments, the surfactants are selected from the group consisting of sulfonates, sulfates, phosphates, carboxylates, and mixtures thereof. In one aspect, the surfactant is selected from (C8-C22)alkyl sulfate salts (e.g., sodium salt); di(C8-C13)alkyl sulfosuccinate salts; (C8-C22)alkyl sarconsinate; (C8-C22)alkyl lactylates; and combinations thereof. Combinations of various surfactants can also be used. Examples of surfactants, and suitable amounts, are disclosed in U.S. Patent Application Publication No. 2013/0190408 (Scholz et al.).

In some embodiments, it is particularly convenient to use a surfactant predissolved in a non-volatile carrier or added along with a carrier. Importantly, the carrier is typically thermally stable and can resist chemical breakdown at processing temperatures which may be as high as 150° C., 180° C., 200° C., or even as high as 250° C. In a preferred embodiment, the surfactant carrier is a liquid at 23° C. Preferred carriers include polyalkylene oxides such as polyethylene glycol, polypropylene glycol, random and block copolymers of ethylene oxide and propylene oxide, thermally stable polyhydric alcohols such as propylene glycol, glycerin, polyglycerin, and the like. The polyalkylene oxides may be linear or branched depending on the initiating polyol. For example, a polyethylene glycol initiated using ethylene glycol would be linear but one initiated with glycerin, trimethylolpropane, or pentaerythritol would be branched. Examples of surfactant carriers, and suitable amounts, are disclosed in U.S. Patent Application Publication No. 2013/0190408 (Scholz et al.).

In some embodiments, the surfactant is soluble in the carrier at extrusion temperatures at the concentrations used. Solubility can be evaluated, for example, as the surfactant and carrier form a visually transparent solution in a 1-cm path length glass vial when heated to extrusion temperature (e.g., 150-190° C.). In some embodiments, the surfactant is soluble in the carrier at 150° C. In some embodiments, the surfactant is soluble in the carrier at less than 100° C. so that it can be more easily incorporated into the polymer melt. In some embodiments, the surfactant is soluble in the carrier at 25° C. so that no heating is necessary when pumping the solution into the polymer melt. In some embodiments, the surfactant is soluble in the carrier at greater than 10% by weight, or greater than 20% by weight, or greater than 30% by weight, in order to allow addition of the surfactant without too much carrier present, which may plasticize the thermoplastic.

In some embodiments, thermoplastic antishrinkage additives incorporated into and/or coated on the polyester-containing fibers include at least one thermoplastic semicrystalline polymer. In some embodiments, the thermoplastic semicrystalline polymer selected from the group consisting of polyethylene, linear low density polyethylene, polypropylene, polyoxymethylene, poly(vinylidine fluoride), poly(methyl pentene), poly(ethylene-chlorotrifluoroethylene), poly(vinyl fluoride), poly(ethylene oxide), poly(ethylene terephthalate), polybutylene terephthalate), semicrystalline aliphatic polyesters including polycaprolactone, aliphatic polyamides such as nylon 6 and nylon 66, and thermotropic liquid crystal polymers. Examples of other suitable antishrink additives, and suitable amounts, are disclosed in U.S. Patent Publication Nos. 2011/0151737 (Moore et al.) and 2011/0151738 (Moore et al.).

Examples of antistatic additives incorporated into and/or coated on the polyester-containing fibers include surfactants such as those listed above as well as cationic and zwitterionic surfactants and hydrophilic polymers. Preferred hydrophilic antistatic polymers are charged (anionic, cationic or zwitterionic). The antistatic additives may be added predissolved in a non-volatile carrier or added along with a carrier.

Examples of colorants (pigments and dyes) incorporated into and/or coated on the polyester-containing fibers include phthalocyanines and inorganic pigments such as titanium dioxide.

Examples of nucleating agents, for increasing crystallinity, include saccharin, talc, boron nitride, ammonium chloride, PHB seed crystals, “polymer soluble” nucleants such as organic phosphonic acids and their combinations with stearic acid salts (see, for example, WO 1991/019759 (Barham et al.)).

Examples of antioxidants incorporated into and/or coated on the polyester-containing fibers include hindered phenols and hindered amines.

Examples of plasticizers incorporated into and/or coated on the polyester-containing fibers include those described in U.S. Pat. No. 6,127,512 (Asrar et al.).

### Polyolefin-Containing Fabric/Film Layer

A polyolefin-containing layer can be in the form of a fabric layer (which may include multiple layers of nonwoven plies) or a film layer (which may include multiple plies), or a combination thereof.

The polyolefin-containing layer includes at least one polyolefin, at least a portion of which is exposed at the surface adjacent to a tie layer that bonds the polyolefin-containing layer to the polyester-containing layer. In certain embodiments, the polyolefin-containing layer has a polyolefin exposed at 50% or more (or at least 60%, or at least 70%, or at least 80%, or at least 90%) of the surface of the layer adjacent to a tie layer that bonds the polyolefin-containing layer to the polyester-containing layer. In certain embodiments, the polyolefin-containing layer includes at least 50% by weight (or at least 60%, or at least 70%, or at least 80%, or at least 90% by weight) of one or more polyolefins.

If the polyolefin-containing layer is in the form of a film layer, suitable film layers include, for example, cast or blown nonporous films. Such nonporous film layers are typically perforated. The film layer material per se typically provides a fluid barrier (e.g., gas and/or liquid), and preferably, the film layer material per se typically provides a gas barrier, particularly, an air barrier. The film layer may include one or more plies. As used herein the term “barrier” refers to a material for making the film layer that does not allow air to pass through the material per se but directs the air through the perforations.

If the polyolefin-containing layer is in the form of a fabric layer, the tie layer material per se typically provides a fluid barrier (e.g., gas and/or liquid), and preferably, the tie layer material per se typically provides a gas barrier, particularly, an air barrier. As used herein the term “barrier” refers to a material for making the tie layer that does not allow air to pass through the material per se but directs the air through the openings of the fabric layer.

As used herein, a “polyolefin layer” or “polyolefin-containing layer” is a fabric or film layer wherein at least 60 weight percent (wt-%) of polymers present in the layer include at least 50 wt-% olefin monomer units. In some embodiments, at least 70 wt-%, or even at least 80 wt-%, polymers in the polyolefin layer include at least 50 wt-% olefin monomer units. In some embodiments, the polymers include at least 70 wt-%, e.g., at least 80 wt-%, or even at least 90 wt-%, olefin monomer units. In some embodiments, at least one polymer consists of olefin monomers. In some embodiments, at least 80 wt-%, in some embodiments, at least 90 wt-%, or even at least 95 wt-%, of the polymers present in the barrier layer consist of olefin monomers.

Exemplary materials suitable for use in the polyolefin-containing layer include polyolefins such as low density polyethylene (LDPE), linear low density polyethylene (LLDPE), metallocene polyethylene, polypropylene (PP), metallocene polypropylene, and the like. Suitable polymers for the film layer also include blends of polyethylenes, blends of polypropylenes, blends of polyethylene and polypropylene, blend polyethylene and/or polypropylene with suitable amorphous polymers, copolymers made from ethylene and propylene monomers, and blends of such copolymers with polyethylenes, polypropylenes, suitable amorphous polymers, semi-crystalline/amorphous polymers, heterophasic polymers, or combinations thereof.

Other useful polymers that may be used in the polyolefin-containing layer include elastomeric thermoplastic polymers. Examples of useful polymers that can be included in the polyolefin-containing layer include those available under the trade names EXXPOL, EXCEED, and EXACT from Exxon Chemical Company of Baytown, Tex.; those available under the trade names ENGAGE, ACHIEVE, ATTAIN, AFFINITY, INFUSE, VERSIFY, and ELITE from Dow Chemical Company of Midland, Mich.

A polyolefin-containing film layer has a thickness that may be varied depending upon the particular end use. In some embodiments, a polyolefin-containing film layer has a thickness of up to 300 micrometers (m), or up to 150 μm, or up to 50 μm, or up to 25 μm, or up to 10 μm. In some embodiments, a polyolefin-containing film layer has a thickness of at least 5 μm, or at least 10 μm, or at least 15 μm, or at least 25 μm. Such thicknesses are measured with essentially no pressure applied, using, for example, an optical comparator. In some embodiments, a polyolefin-containing film layer has a thickness of from 10 μm to about 50 μm. For use in some applications such as medical fabrics, including disposable warming blankets and gowns, the thickness of a polyolefin-containing film layer is typically from 5 μm to 25 μm.

Polyolefin-containing layers may have similar physical properties (e.g., fiber diameter, basis weight, thickness) as the polyester-containing layer described above.

### Tie Layer

Despite the individual advantages associated with the polyester-containing layer and the polyolefin-containing layer, bonding these layers to each other to form the desired finished laminate for use in an inflatable medical article, particularly one that must withstand elevated temperatures and pressures, as in a warming blanket, was not previously known in the art.

Tie layer compositions for good adhesion between a polyester-containing layer (e.g., a nonwoven fabric of an aliphatic polyester, such as Polylactide (PLA)) and a polyolefin-containing layer (e.g., a film or nonwoven fabric) contain copolymers of ethylene and methyl acrylates, for example. Examples of such tie layer compositions are disclosed in International Publication No. WO 2014/059239. These copolymers are often denoted as Ethylene Methyl Acrylate Copolymer (EMAC) and thus they contain a certain percentage of methyl acrylate (MA) entities as comonomer in the polymer chains. Higher contents of MA allow better adhesion with polyester nonwovens which is important for ensuring high sealing forces with the laminates and other added films in the composite, such as in the patient warming products. Higher MA content (e.g., greater than 22 wt-%), however, also leads to low melting points, low Vicat softening point, and/or low glass transition temperatures. For example, LOTRYL 24MA02, which includes 24% MA, available from Arkema Functional Polyolefins of Colombes, France, has a Vicat softening point of 49° C. per ASTM D1525. EMACs with low melting points and/or low Vicat softening temperatures, while showing good adhesion between such materials, become soft when exposed to heated surfaces of hot air. This heating leads to softening of the polymer-fiber interface leading to release of nonwoven fibers from the film layer.

The present disclosure provides a tie layer that includes a copolymer having a Vicat softening temperature of greater than 45° C. (and in certain embodiments, at least 50° C., or at least 60° C.) that can be used to bond the fabric layer to the film layer. Such copolymers can be prepared from monomers that include at least one olefin monomer and at least 2 wt-%, or at least 5 wt-% (in certain embodiments, at least 7 wt-%) of one or more polar monomers. In some embodiments, the copolymer of the tie layer is prepared from monomers that include up to 22 wt-%, or up to 20 wt-%, or up to 18 wt-%, or up to 15 wt-%, of one or more polar monomers.

Exemplary polar monomers include vinyl acetate (VA); (C1-C8)alkyl esters of (meth)acrylic acid (i.e., acrylates and methacrylates) such as ethyl acrylate (EA), methyl acrylate (MA), butyl acrylate (BA), and 2-ethylhexyl acrylate; and (C1-C4)(meth)acrylic acids (e.g., acrylic acid and methacrylic acid). Exemplary copolymers include EVA copolymers available from Dupont Company under the trade name ELVAX, e.g., ELVAX3170, from CELENASE under the trade name ATEVA, e.g., ATEVA 1240A, and from LANXESS GMBH under the trade name LEVAMELT, e.g., LEVAMELT 450, and methyl acrylate ethylene copolymers (EMA) such as those available under the trade name ELVALOY from Dupont, such as ELVALOY AC 1609, ELVALOY AC 1913.

In some embodiments, the copolymer of the tie layer further includes at least one reactive monomer. In some embodiments, the reactive monomer includes a reactive group that is capable of reacting with, and covalently bonding to, a hydroxyl group, such as the terminal groups of an aliphatic polyester. Such reaction is capable of occurring at elevated temperatures that can be reached during extrusion, such as a temperature of at least 150° C., or at least 175° C., or at least 200° C., or at least 225° C. Exemplary reactive groups include anhydride, active ester, epoxy, isocyanate, azalactone, carboxylic acid halides, and combinations thereof.

In some embodiments, the tie layer is a copolymer including at least three different monomers: an olefin monomer (e.g., ethylene); a polar nonreactive monomer (e.g., vinyl acetate or a methacrylate monomer); and a reactive monomer (e.g., a monomer having an anhydride or epoxy group). In some embodiments, the copolymer of the tie layer includes greater than 0.1 wt-%, or greater than 0.5 wt-%, or greater than 1 wt-%, or even greater than 2 wt-%, of reactive monomer, and greater than 5 wt-%, or greater than 10 wt-%, or even greater than 12 wt-%, or nonreactive polar monomer.

In some embodiments, the tie layer further includes a reactive polymer having at least one reactive monomer. In some embodiments, the reactive monomer includes a reactive group that is capable of reacting with, and covalently bonding to, a hydroxyl group, such as the terminal groups of an aliphatic polyester. Such reaction is capable of occurring at elevated temperatures that can be reached during extrusion, such as a temperature of at least 150° C., or at least 175° C., or at least 200° C., or at least 225° C. Exemplary reactive groups include anhydride, active ester, epoxy, isocyanate, azalactone, carboxylic acid halides, and combinations thereof.

Exemplary reactive tie layer copolymers include those available under the trade name TYMAX from Westlake Chemical Corp., Houston, Tex. (e.g., TYMAX GA7001 which is believed to be a terpolymer of 20% methyl acrylate, ethylene, and an anhydride containing reactive monomer) and those available under the trade name LOTADUR from Arkema (e.g., LOTADUR TX8030 which is a maleic anhydride terpolymer of 13% ethyl acrylate, 2.8% maleic anhydride content and 84.2% ethylene, and LOTADUR AX8900, which is believed to be a terpolymer of 24% methylacrylate, 8% glycidylmethyacrylate and 68% ethylene.

Small amounts of polymers that have lower Vicat softening temperatures (e.g., LOTADUR AX8900 has a Vicat softening temperature of less than 40° C.) may be included in some embodiments, as long as the entire tie layer composition has a Vicat softening temperature of greater than 45° C.

In some embodiments, the polymeric composition of the tie layer may include one or more plasticizers. Exemplary plasticizers include alkyl benzoates such as those available under the trade name FINSOLV from Innospec Performance Chemicals.

In some embodiments, the polymer composition of the tie layer may include one or more tackifiers. A wide variety of resinous (or synthetic) materials commonly used in the art to impart or enhance adhesion of the tie layer to the aliphatic polyester nonwoven-containing layer and to the polyolefin film layer may be used as a tackifier. In some embodiments, the tackifiers have a ring and ball softening point greater than 90° C. and in some embodiments, greater than 100° C. according to ASTM E 28 and a weight average molecular weight of greater than 700 and in some embodiments, greater than 800 or even greater than 900 g/mol.

Exemplary tackifiers include rosin, rosin esters of glycerol or pentaerythritol, hydrogenated rosins, polyterpene resins such as polymerized beta-pinene, coumaroneindene resins, “C5” and “C9” polymerized petroleum fractions, and the like. Suitable commercially available tackifiers include synthetic ester resins, such as that available under the trade name FORAL (e.g., FORAL 85) from Hercules Inc., Wilmington, Del., and aliphatic/aromatic hydrocarbon resins, such as those available under the trade name ESCOREZ (e.g., ESCOREZ 5690) from Exxon Chemical Co., Houston, Tex. and REGALREZ (e.g., REGALREZ 6108 and 3102) from Eastman Chemical Company Kingsport, Tenn.

Generally, the tackifier is added in amounts required to achieve the desired tack, adhesion, and/or coefficient of friction level. This is typically achieved by adding from 1 part to 100 parts by weight of tackifier per 100 parts by weight of the tie layer copolymer. In some embodiments, the tackifier is added at from 2 to 20 parts by weight tackifier to 100 parts by weight of the tie layer copolymer. The tackifier is selected to provide the tie layer polymers with an adequate degree of tack while molten to promote adhesion and to maintain their adhesion when cooled. As is known in the art, not all tackifier resins interact with the tie layer polymer in the same manner; therefore, some minor amount of experimentation may be required to select the appropriate tackifier resin and concentration to achieve the desired adhesive performance. Such minor experimentation is well within the capability of one skilled in the adhesive art.

If the polyolefin-containing layer is in the form of a fabric layer, the tie layer material may be in the form of a film or sheet, wherein, the tie layer material per se typically provides a fluid barrier (e.g., gas and/or liquid), and preferably, the tie layer material per se typically provides a gas barrier, particularly, an air barrier. As used herein the term “barrier” refers to a material for making the tie layer that does not allow air to pass through the material per se but directs the air through the openings of the fabric layer. This means that air at a pressure of 5 cm of water is able to inflate a 7.5 cm diameter×1 meter long tube to a level of at least 60% inflation from a blower such as 3M BAIR HUGGER model 500 and 700 series warming units.

### Sheet Bonded to Laminate

Generally, any of a wide variety of materials may be used to form the sheet that is bonded to the laminate to form at least one inflatable chamber. In some embodiments, the sheet may be made of the same material as the film layer or the laminate, as described herein. The film material can be either single layer or multilayer cast or blown film. Preferred film sheets are made of polyoelfins such as low density polyethylene (LDPE), linear low density polyethylene (LLDPE), metallocene polyethylene, polypropylene (PP), metallocene polypropylene, and the like. Suitable polymers for the sheet layer also include blends of polyethylenes, blends of polypropylenes, blends of polyethylene and polypropylene, blend polyethylene and/or polypropylene with suitable amorphous polymers, copolymers made from ethylene and propylene monomers, and blends of such copolymers with polyethylenes, polypropylenes, suitable amorphous polymers, semi-crystalline/amorphous polymers, heterophasic polymers, or combinations thereof.

### Optional Components for One or More Layers

Other optional components may be included in one or more layers of the articles described herein. For example, in some embodiments, an antimicrobial component may be added to impart antimicrobial activity. The antimicrobial component is that component that provides at least part of the antimicrobial activity, i.e., it has at least some antimicrobial activity for at least one microorganism. It is preferably present in a large enough quantity to be leached out and kill bacteria or to kill on contact without leaching. It may also be biodegradable and/or made or derived from renewable resources such as plants or plant products. Biodegradable antimicrobial components can include at least one functional linkage such as an ester or amide linkage that can be hydrolytically or enzymatically degraded.

Examples of antimicrobial components suitable for use in the articles of the present disclosure include those described in Applicants' co-pending application, U.S. Patent Application Publication No. 2008/0142023 (Schmid et al.). Certain antimicrobial components are uncharged and have an alkyl or alkenyl hydrocarbon chain containing at least 7 carbon atoms. For melt processing, preferred antimicrobial components have low volatility and do not decompose under process conditions. The preferred antimicrobial components contain less than 2 wt-% water, and more preferably less than 0.10 wt-% (determined by Karl Fischer analysis). Moisture content is kept low in order to prevent hydrolysis of the aliphatic polyester and to give clarity to extruded film. Certain antimicrobial components are amphiphiles and may be surface active. For example, certain antimicrobial alkyl monoglycerides are surface active. Certain cationic antimicrobial amine compounds also may be useful as described in U.S. Patent Application Publication No. 2008/0142023 (Schmid et al.).

When used, the antimicrobial component content (as it is ready to use) is typically at least 1 wt.-%, at least 2 wt-%, at least 5 wt-%, or at least 10 wt-%, and sometimes greater than 15 wt-%. In certain embodiments, in which a low strength is desired, the antimicrobial component content is typically greater than 20 wt-%, greater than 25 wt-%, or even greater than 30 wt-%. The antimicrobial component may be predissolved in or added along with a carrier to enhance activity.

In some embodiments, one or more of the layers may further include organic and inorganic fillers. In some embodiments, biodegradable, resorbable, or bioerodible inorganic fillers may be particularly appealing. These materials may help to control the degradation rate of polymers. For example, many calcium salts and phosphate salts may be suitable. Exemplary biocompatible resorbable fillers include calcium carbonate, calcium sulfate, calcium phosphate, calcium sodium phosphates, calcium potassium phosphates, tetracalcium phosphate, .alpha.-tricalcium phosphate, beta-tricalcium phosphate, calcium phosphate apatite, octacalcium phosphate, dicalcium phosphate, calcium carbonate, calcium oxide, calcium hydroxide, calcium sulfate dihydrate, calcium sulfate hemihydrate, calcium fluoride, calcium citrate, magnesium oxide, and magnesium hydroxide. A particularly suitable filler is tribasic calcium phosphate (hydroxy apatite).

In some embodiments, plasticizers may be used with the aliphatic polyester thermoplastic and include, for example, glycols such glycerin; propylene glycol, polyethoxylated phenols, mono or polysubstituted polyethylene glycols, higher alkyl substituted N-alkyl pyrrolidones, sulfonamides, triglycerides, citrate esters, esters of tartaric acid, benzoate esters, polyethylene glycols and ethylene oxide propylene oxide random and block copolymers having a molecular weight less than 10,000 daltons preferably less than about 5000 daltons, more preferably less than about 2500 daltons; and combinations thereof.

Other additional components include antioxidant, colorant such as dyes and/or pigments, antistatic agents, fluorescent brightening agents, odor control agents, perfumes and fragrances, active ingredients to promote wound healing or other dermatological activity, combinations thereof, and the like.

### Preparation of Articles

Patient warming devices that may include the embodiments of the present disclosure may be formed by joining two sheets of material with a closed impermeable seam formed by sealing the sheets of material around their peripheries and, in some embodiments, one or more additional closed impermeable seams to define separate inflatable sections. Typically, one of the sheets is relatively impermeable and the other sheet is relatively more permeable to permit airflow therethrough. A sheet can be air permeable using various materials or mechanical structures, for example, air-permeable materials, apertures, interstices, slits, or the like. One or more inlet ports may be provided for introducing warm air to inflate the device and warm the patient or clinician by convective warming via the distributed warm air. Unused inlet ports are sealed or closed by known means to prevent air escaping therethrough. Preferably the inlet port is provided through the impermeable surface/layer of the convective warming apparatus. The inlet port may comprise a collar of stiff material mounted on a portion of the impermeable surface in the section with an opening through the surface to receive the nozzle of an air hose of a heater blower unit, or it may comprise a sleeve of material, or any other equivalent structure. In some embodiments of the inflatable sections, the permeability of the permeable surface may vary in different portions of the sections (e.g., the upper section, middle section, low sections, etc.) in order to reduce or eliminate variances in temperature of air expelled through the permeable surface of the section. A warming device may have one or more convective apparatuses and each convective apparatus may have one or more inflatable sections.

Air permeable materials include, for example, woven fabrics, nonwoven fabrics, perforated film, porous film, laminated material (e.g, nonwoven fabrics with perforated film, etc.), flocked fabrics, and the like. Nonwoven fabrics include, for example, carded thermally bonded nonwovens, spunbond nonwovens, hydroentangled/spunlaced nonwovens, SMS (Spunbond-Meltblown-Spunbond) nonwovens, air-laid nonwovens, wet-laid nonwovens, or the like. The air impermeable strip uses materials having less air permeability (i.e., air impermeable materials).

Air impermeable materials include, for example, single layer plastic film (e.g., polyethylene, propylene, polyurethane, polyester, etc.), metal film (e.g., aluminum foil film, etc.), elastic film (e.g., polyurethane, Kratons, etc.), multi-layer film (e.g., co-extruded film, blown film, etc.), film coated paper, and the like. In some implementations of an air permeable sheet with apertures, the density of apertures can vary among areas and/or inflatable sections. Furthermore the sheets may be connected by discontinuous seals or stake points within the closed impermeable seams. The two sheets with which a convective apparatus is formed may be separate from a clinical garment, in which case the convective apparatus may be permanently or releasably attached, fixed, or adhered to the inside surface of the clinical garment with permeable surfaces facing inwardly, toward a patient. An exemplary construction in this regard is illustrated in FIGS. 1A and 1D and FIGS. 3A-3C of International Pub. No. WO 2003/086500 (Augustine et al.).

### Exemplary Embodiments

Embodiment 1 is an inflatable medical article comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer to the polyolefin-containing layer;
wherein the tie layer comprises a copolymer prepared from monomers comprising at least one olefin monomer and up to 22 wt-% of at least one polar monomer, wherein the copolymer has a Vicat softening temperature of greater than 45° C.;
an optional sheet; and
at least one inflatable chamber formed between the polyolefin-containing layer and the tie layer, or between the polyester-containing layer and the tie layer, or between the polyolefin-containing layer and the optional sheet when such sheet is present.

Embodiment 2 is the inflatable medical article of embodiment 1 further comprising a plurality of engineered openings for fluid communication between the at least one inflatable chamber and the environment.

Embodiment 3 is the inflatable medical article of embodiment 1 or 2 wherein the at least one inflatable chamber is formed between the polyolefin-containing layer and the tie layer.

Embodiment 4 is the inflatable medical article of embodiment 1 or 2 wherein the at least one inflatable chamber is formed between the polyester-containing layer and the tie layer.

Embodiment 5 is an inflatable medical article comprising:
a laminate comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer to the polyolefin-containing layer;
wherein the tie layer comprises a copolymer prepared from monomers comprising at least one olefin monomer and up to 22 wt-% of at least one polar monomer, wherein the copolymer has a Vicat softening temperature of greater than 45° C.; and
a sheet bonded to the laminate to create at least one inflatable chamber.

Embodiment 6 is the inflatable medical article of embodiment 5 wherein the polyolefin-containing layer is a film layer.

Embodiment 7 is the inflatable medical article of embodiment 5 or 6 further comprising a plurality of engineered openings for fluid communication between the at least one inflatable chamber and the environment.

Embodiment 8 is the inflatable medical article of any one of embodiments 5 through 7 wherein the sheet is bonded to the polyolefin-containing layer of the laminate.

Embodiment 9 is the inflatable medical article of any one of embodiments 5 through 8 wherein the tie layer has a Vicat softening temperature of greater than 45° C.

Embodiment 10 is the inflatable medical article of any one of embodiments 5 through 9 wherein the polyester-containing layer is fluid repellent.

Embodiment 11 is the inflatable medical article of any one of embodiments 5 through 10 wherein the polyester-containing layer is a first polyester-containing layer, the polyolefin-containing layer has a first major surface and a second major surface, and the tie layer is a first tie layer bonding the first polyester-containing layer to the first major surface of the polyolefin-containing layer, and the laminate further comprises:
a second polyester-containing layer comprising a fabric layer comprising at least one nonwoven web of fibers comprising an aliphatic polyester, wherein at least a portion of the aliphatic polyester is exposed at the surface of the fibers; and
a second tie layer bonding the first polyester-containing layer to the second major surface of the polyolefin-containing layer;
wherein the second tie layer comprises a copolymer prepared from monomers comprising at least one olefin monomer and up to 22 wt-% of at least one polar monomer, wherein the copolymer has a Vicat softening temperature of greater than 45° C.

Embodiment 12 is the inflatable medical article of any one of embodiments 5 through 11 wherein the polyester-containing layer comprises two or more nonwoven webs of fibers comprising an aliphatic polyester, wherein at least a portion of the aliphatic polyester is exposed at the surface of the fibers.

Embodiment 13 is the inflatable medical article of any one of embodiments 5 through 12 wherein the laminate is a first laminate, and the sheet is in the form of a second laminate comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer of the second laminate to the polyolefin-containing layer of the second laminate;
wherein the tie layer comprises a copolymer prepared from monomers comprising at least one olefin monomer and up to 22 wt-% of at least one polar monomer, wherein the copolymer has a Vicat softening temperature of greater than 45° C.

Embodiment 14 is the inflatable medical article of embodiment 13 wherein the polyester-containing layer of the second laminate comprises two or more nonwoven webs of fibers comprising an aliphatic polyester, wherein at least a portion of the aliphatic polyester is exposed at the surface of the fibers.

Embodiment 15 is the inflatable medical article of any one of embodiments 5 through 14 which can withstand inflation at a pressure of 2.0 inches water (50 mm water) with air at a temperature greater than 38° C. without separation of the polyester-containing layer from the polyolefin-containing layer.

Embodiment 16 is the inflatable medical article of any one of embodiments 5 through 15 wherein the tie layer further comprises a tackifier.

Embodiment 17 is the inflatable medical article of any one of embodiments 5 through 16 wherein at least 50% of the surface area of the exterior surface area of the fibers of the polyester-containing layer and/or at least 50% of the surface area of the nonwoven of the polyester-containing layer includes an aliphatic polyester.

Embodiment 18 is the inflatable medical article of embodiment 17 wherein at least 75% of the surface area of the exterior surface area of the fibers of the polyester-containing layer and/or at least 75% of the surface area of the nonwoven of the polyester-containing layer includes an aliphatic polyester.

Embodiment 19 is the inflatable medical article of any one of embodiments 5 through 18 wherein the tie layer comprises a copolymer prepared from monomers comprising at least one olefin monomer and at least 7 wt-% of at least one polar monomer.

Embodiment 20 is the inflatable medical article of embodiment 19 wherein the tie layer comprises a copolymer prepared from monomers comprising one or more olefin monomers and up to 20 wt-% of one or more polar monomers.

Embodiment 21 is the inflatable medical article of embodiment 20 wherein the tie layer comprises a copolymer prepared from monomers comprising one or more olefin monomers and up to 18 wt-% of one or more polar monomers.

Embodiment 22 is the inflatable medical article of embodiment 21 wherein the tie layer comprises a copolymer prepared from monomers comprising one or more olefin monomers and up to 15 wt-% of one or more polar monomers.

Embodiment 23 is the inflatable medical article of any one of embodiments 5 through 22 wherein the at least one olefin monomer of the copolymer of the tie layer is ethylene.

Embodiment 24 is the inflatable medical article of any one of embodiments 5 through 23 wherein the tie layer comprises a copolymer prepared from monomers comprising at least one olefin monomer and up to 22 wt-% of at least one polar monomer, wherein the copolymer has a Vicat softening temperature of greater than 55° C.

Embodiment 25 is the inflatable medical article of embodiment 24 wherein the tie layer comprises a copolymer prepared from monomers comprising at least one olefin monomer and up to 22 wt-% of at least one polar monomer, wherein the copolymer has a Vicat softening temperature of greater than 60° C.

Embodiment 26 is the inflatable medical article of any one of embodiments 5 through 25 wherein the at least one polar monomer is selected from vinyl acetate, a (C1-C8)alkyl ester of (meth)acrylic acid, a (C1-C4)(meth)acrylic acid, and combinations thereof.

Embodiment 27 is the inflatable medical article of embodiment 26 wherein the at least one polar monomer is vinyl acetate or methyl acrylate.

Embodiment 28 is the inflatable medical article of any one of embodiments 5 through 27 wherein the polyolefin-containing layer comprises a polyolefin selected from low density polyethylene (LDPE), linear low density polyethylene (LLDPE), metallocene polyethylene, polypropylene (PP), metallocene polypropylene, and combinations thereof.

Embodiment 29 is the inflatable medical article of any one of embodiments 5 through 28 which is in the form of a blanket, a pad, or a garment.

Embodiment 30 is an inflatable medical article comprising:
a polyester-containing layer comprising a fabric layer comprising a nonwoven web of fibers comprising an aliphatic polyester, wherein at least a portion of the aliphatic polyester is exposed at the surface of the fibers;
a polyolefin-containing film layer; and
a tie layer bonding the polyester-containing layer to the polyolefin-containing layer;
wherein the tie layer comprises a copolymer having a Vicat Softening Temperature of at least 50° C., which is prepared from monomers comprising at least one olefin monomer and at least one polar monomer in an amount of 5-18 wt-%;
wherein the inflatable medical article can withstand inflation at a pressure of 2.0 inches water (50 mm water) with air at a temperature greater than 38° C. without separation of the fabric layer from the film layer; and
a sheet bonded to the polyolefin-containing film layer to create at least one inflatable chamber.

Embodiment 31 is the inflatable medical article of embodiment 30 wherein the tie layer comprises a copolymer prepared from monomers comprising one or more olefin monomers and 7-15 wt-% of one or more polar monomers.

Embodiment 32 is the inflatable medical article of embodiment 31 wherein the at least one olefin monomer of the copolymer of the tie layer is ethylene.

Embodiment 33 is the inflatable medical article of embodiment 32 wherein the at least one polar monomer is selected from vinyl acetate, a (C1-C8)alkyl ester of (meth)acrylic acid, a (C1-C4)acrylic acid, and combinations thereof.

Embodiment 34 is the inflatable medical article of embodiment 33 wherein the at least one polar monomer is vinyl acetate or methyl acrylate.

Embodiment 35 is a patient warming device comprising the inflatable medical article of any one of embodiments 1 through 34 and a convective apparatus integrated with or attached to the inflatable medical article.

### Examples

Objects and advantages of this invention are further illustrated by the following examples, but the particular materials and amounts thereof recited in these examples, as well as other conditions and details, should not be construed to unduly limit this invention. These examples are merely for illustrative purposes only and are not meant to be limiting on the scope of the appended claims.

TABLE 1
Summary of Materials Used in the Examples

| I.D. | Description | Source (Trade name) |
|---|---|---|
| PLA | Polylactic acid | Natureworks LLC, Blair, |
| | | Nebraska |
| | | (INGEO 6202D) |
| LDPE | Low density polyethylene | Chevron Phillips |
| | | Chemical Company LP, |
| | | The Woodlands, |
| | | Texas (MARFLEX 1019) |
| LLDPE | Linear low density polyethylene | DOW Chemical Company |
| | | (DOWLEX 2517) |
| Tack-1 | 100% hydrogenated C9 | Eastman Chemical, |
| | aromatic resin used | Kingsport, Tennessee |
| | as a tackifier | (REGALREZ 6108) |
| EMA-24 | Ethylene-methyl acrylate | Dupont, Wilmington, |
| | copolymer with | Delaware |
| | 24% methyl acrylate comonomer | (ELVALOY AC 12024S) |
| EMA-20 | Ethylene-methyl acrylate | Dupont, Wilmington, |
| | copolymer with | Delaware |
| | 20% methyl acrylate comonomer | (ELVALOY AC 1820) |
| EMA-18 | Ethylene-methyl acrylate | Dupont, Wilmington, |
| | copolymer with | Delaware |
| | 18% methyl acrylate comonomer | (ELVALOY AC 1218) |
| EMA-13 | Ethylene-methyl acrylate | Dupont, Wilmington, |
| | copolymer with | Delaware |
| | 13% methyl acrylate comonomer | (ELVALOY AC 1913) |
| EMA-9 | Ethylene-methyl acrylate | Dupont, Wilmington, |
| | copolymer with | Delaware |
| | 9% methyl acrylate comonomer | (ELVALOY AC 1609S) |
| RxAH-1 | Anyhydride modified | Westlake Chemical |
| | ethylene-methyl acrylate | Corporation, |
| | (terpolymer of 20% methyl | Houston Texas (TyMax |
| | acrylate, ethylene, and an anhydride | GA7001) |
| | containing reactive monomer) | |
| EVA-18 | Ethylene vinyl acetate | DuPont Company, |
| | (EVA) copolymer with | Willmington, |
| | 18% vinyl acetate (VA) content | Delaware (ELVAX 3170) |
| EVA-12 | Ethylene vinyl acetate | Celenase; Edmonton, AB |
| | (EVA) copolymer with | (ATEVA 1240A) |
| | 12% vinyl acetate (VA) content | |
| EA-13 | Ethylene-Acrylic ester- | Arkema Technical |
| | Maleic Anhydride | Polymers |
| | Terpolymer, 13% ethyl | Division, Colombes |
| | acrylate content, | Cedex, France |
| | 2.8% maleic anhydride content | (LOTADER TX8030) |
| Blue | Blue pigment in polylactic acid | Techmer Polymer |
| MB1 | | Modifiers |

Multilayer laminate articles were prepared using a PLA-based nonwoven layer. A tie-layer was extrusion coated onto the nonwoven layer. The nonwoven layer was a single layer made using one spunbond beam. The nonwoven was produced using PLA and 0.15% by weight Blue MB1 with a coating basis weight of 38.5 grams per square meter (gsm). A Bench 300 Haake Single Extruder was used to coat the tie layer on the nonwoven. The screw was set at 60 revolutions per minute (rpm) with a line speed of 6.86 meters (22.5 feet) per minute and a coating weight 25-27 gsm. The extruder and die temperatures used for the coating were 182-224° C. (360-435° F.). Nip pressure was 207-276 kPa (30-40 PSI). The Vicat softening temperature, melt flow index (MFI), melt temperature, and amount of polar component of the ingredient in the tie layer are listed in Table 2. The tie-layer formulations used for the additional examples are listed in Tables 3-5.

TABLE 2
Properties of the Tie layer Ingredients

| | | MFI g/10 | Vicat | Melt |
|---|---|---|---|---|
| | Polar | min | Softening | Temp. |
| Ingredient | Component | 190/2.16 kg | Temp. (° C.) | (° C.) |
| EMA-24 | 24% (MA) | 20 | 45 | 88 |
| EMA-20 | 20% (MA) | 8 | 54 | 92 |
| EMA-18 | 18% (MA) | 2 | 60 | 94 |
| EMA-13 | 13% (MA) | 9 | 60 | 98 |
| EMA-9 | 9% (MA) | 6 | 70 | 103 |
| EA-13 | 13% (EA) | 3 | 65 | 95 |
| EVA-18 | 18% (VA) | 2.5 | 65 | 87 |
| EVA-12 | 12% (VA) | 10 | 72 | 97 |
| RxAH-1 | 20% (MA) | 6 | ** | 95 |
| LDPE | 0% | 16 | 87 | 103 |

**Not Available.

TABLE 3
Summary of Tie-layer Formulations Examples
EX-1 to EX-5

| Tie layer | EX-1 | EX-2 | EX-3 | EX-4 | EX-5 |
|---|---|---|---|---|---|
| EMA-9 | 81% | — | — | — | — |
| EMA-13 | — | 81% | — | — | — |
| EMA-18 | — | — | 81% | — | — |
| EMA-20 | — | — | — | 81% | — |
| EMA-24 | — | — | — | — | 81% |
| RxAH-1 | 10% | 10% | 10% | 10% | 10% |
| Tack-1 | 9% | 9% | 9% | 9% | 9% |
| Wt % of MA | 9.3 | 12.5 | 16.6 | 18.2 | 21.4 |
| in tie layer | | | | | |

TABLE 4
Summary of Tie-layer Formulations Examples
EX-6 to EX-10

| Tie layer | EX-6 | EX-7 | EX-8 | EX-9 | EX-10 |
|---|---|---|---|---|---|
| EMA-9 | 35% | 41% | 25% | 90% | 51% |
| EMA-13 | — | — | — | — | — |
| EMA-18 | — | 40% | — | — | — |
| EMA-24 | 46% | — | 56% | — | — |
| RxAH-1 | 10% | 10% | 10% | 10% | 10% |
| Tack-1 | 9% | 9% | 9% | — | 9% |
| LDPE | — | — | — | — | 30% |
| Wt % of MA | 16.2 | 12.9 | 17.7 | 10.1 | 6.6 |
| in tie layer | | | | | |

TABLE 5
Summary of Tie-layer Formulations Examples
EX-11 to EX-14

| Tie layer | EX-11 | EX-12 | EX-13 | EX-14 |
|---|---|---|---|---|
| RxAH-1 | 10% | 10% | 10% | 10% |
| Tack-1 | 9% | 9% | 9% | 9% |
| LDPE | 81% | — | — | — |
| EA-13 | — | 81% | — | — |
| EVA-18 | — | — | 81% | — |
| EVA-12 | — | — | — | 81% |
| Wt % of MA in tie layer | 2.0 | 2.0 | 2.0 | 2.0 |
| Wt % of EVA in tie layer | — | — | 14.6 | 9.7 |
| Wt % of EA in tie layer | — | 10.5 | — | — |

Peel Testing of Seals Formed with Examples EX-1-EX-14

Examples EX-1-EX-14 were peel tested in the machine direction (MD) and the cross direction (CD) using samples cut to 2.5 cm (1 inch) wide and 10.2 cm (4 inch) long under environmental condition of 43° C. using Zwick/Roell Model Z005 Tensile Tester. A gauge length of 2.54 cm and test speed of 304 mm/min were used for the tests. Before testing, the samples were first prepared by adding two equal sized pieces of SCOTCH Premium Heavy Duty Packaging Tape 3750 Clear-to-Core (available from 3M Company of St. Paul, Minn.), one piece of the tape applied to reinforce the nonwoven side and the second piece applied to the tie layer side. Peel was initiated between the tie layer and the nonwoven substrate. The results are listed in the Table 6.

TABLE 6
Peel Test Results at 43° C.

| | AVERAGE FORCE (N) | | | | MAX FORCE (N) | | | |
|---|---|---|---|---|---|---|---|---|
| | CD | | MD | | CD | | MD | |
| | FORCE | STD | FORCE | STD | FORCE | STD | FORCE | STD |
| EXAMPLE | (N) | DEV | (N) | DEV | (N) | DEV | (N) | DEV |
| EX-1 | 3.02 | 0.32 | 4.77 | 0.28 | 4.9 | 0.72 | 5.84 | 0.4 |
| EX-2 | 2.6 | 0.32 | 3.4 | 0.32 | 4.26 | 0.58 | 3.8 | 0.31 |
| EX-3 | 1.81 | 0.36 | 2.34 | 0.15 | 3.47 | 0.42 | 3.09 | 0.6 |
| EX-4 | 1.31 | 0.16 | * | * | 1.92 | 0.18 | * | * |
| EX-5 | 1.55 | 0.24 | * | * | 1.64 | 0.31 | * | * |
| EX-6 | 2.8 | 0.22 | * | * | 3.09 | 0.18 | * | * |
| EX-7 | 4.34 | 0.77 | 3.91 | 0.37 | 4.71 | 0.8 | 4.85 | 0.71 |
| EX-8 | 1.5 | 0.18 | * | * | 2.68 | 0.23 | * | * |
| EX-9 | 0.37 | 0.07 | 0.36 | 0.13 | 0.69 | 0.13 | 0.65 | 0.22 |
| EX-10 | 2.92 | 0.28 | 3.73 | 0.52 | 5.63 | 0.71 | 4.51 | 0.82 |
| EX-11 | 2.52 | 0.14 | 2.22 | 1.36 | 5.13 | 0.83 | 2.96 | 1.72 |
| EX-12 | 3.89 | 0.2 | 3.49 | 0.99 | 3.97 | 0.17 | 3.95 | 0.82 |
| EX-13 | 2.23 | 0.45 | 2.7 | 0.21 | 2.66 | 0.5 | 3.3 | 0.4 |
| EX-14 | 1.85 | 0.24 | 0.62 | 0.52 | 3.67 | 0.51 | 2.33 | 0.8 |

Note:
*not tested

### Examples EX-1B, EX-5B, and Comparative Examples C-EX.1-C-EX.6

A PLA spunbond nonwoven was produced by extruding 98.6% PLA 6202D (INGEO, Natureworks), 1% polypropylene (PP3866, Total Petrochemical) and 0.4% of light blue color concentrate in polypropylene (Techmer PPM 56160) at 238° C. (460° F.) on sheath side and 99% PLA 6202D, 1% polypropylene (PP3866, Total Petrochemical) on the core side by extruding at about 238° C. (460° F.). There were two spunbonded fiber dies each extruding 567-612 Kg (1250-1350 pounds) per hour. The extruded fibers were drawn sufficiently such that filament size was measured at an average of 12 micrometer (μm) range consistently. Further downstream the nonwoven was bonded at a temperature of 154-157° C. (310-315° F.) and 354-589 Kg per linear centimeter (300-500 pound per linear inch (pli)) of pressure at 146-174 meters (160-190 yards) per minute for a basis weight of approximately 34 grams per square meter (gsm). This nonwoven was tightly wound in a 2.49 meter wide roll using common winding equipment. The resulting nonwoven demonstrated machine direction (MD) and cross direction (CD) peak tensile load of 50-85 N/5 cm and 15-34 N/5 cm at % elongation of 15-24% in MD and 18-25% in CD respectively. Subsequently and immediately before film forming process (described below), the above nonwoven was corona treated at 25 kW of power to enhanced adherence with the film to the nonwoven.

A tie layer film and a polyethylene (PE) barrier layer film were coextruded together onto the above described PLA spunbond nonwoven. The layers were then run through a 65-70D durometer nip roll and pressed by a water cooled chilled roll at about 26.7° C. (80° F.). The film was dropped from a film die located 20 cm above, and at an offset of 5 cm upstream from the nip line at speeds of about 274 meters (900 feet) per minute. The total weight of the extruded construction was 35% tie layer and 65% barrier layer. The Extruder B extruded the tie layer polymer at a melt temperature of 249-254° C. (480-490° F.) with a recipe comprising of Tack-1, RxAH-1 (optional), and the remaining amount being EMA-9 or EMA-24 as described in Table 7. For Examples EX-1B, EX-5B, and EX-15, the Extruder A extruded a PE barrier film layer polymer at a melt temperature of 254-266° C. (490-510° F.) with a recipe comprising of a combination of 69% LLDPE; 25% LDPE, and remaining 6% being a LDPE based color masterbatch (TECHMER PM 56017) of light blue (Phthalocyanine Blue) and white (TiO₂) pigment, referred to as PE Barrier Film “X” in Table 7. For Examples EX-16 through EX-20, the Extruder A extruded a PE barrier film layer that was 65% LDPE, 29% LLDPE, and remaining 6% being a LDPE based color masterbatch (TECHMER PM 56017) of light blue (Phthalocyanine Blue) and white (TiO₂) pigment, referred to as PE Barrier Film “Y” in Table 7. The coextruded film nonwoven laminates were tightly rolled using common winding equipment.

TABLE 7
Tie-layer + PE Barrier Film + Nonwoven Examples: EX-1B, EX-5B,
and Comparative Examples C-EX.1-C-EX.6

| Tie layer | | | | | | | | |
|---|---|---|---|---|---|---|---|---|
| (35%) | | | Comp | Comp | Comp | Comp | Comp | Comp |
| Composition | EX-1B | EX-5B | EX.1 | EX.2 | EX.3 | EX.4 | EX.5 | EX.6 |
| EMA-9 | 81% | — | — | — | — | — | — | — |
| EMA-24 | — | 81% | 95% | 95% | 95% | 95% | 95% | 95% |
| RxAH-1 | 10% | 10% | — | — | — | — | — | — |
| Tack-1 | 9% | 9% | 5% | 5% | 5% | 5% | 5% | 5% |
| Coating Wt. | 13.8 | 13.2 | 13.8 | 16.8 | 14.6 | 23.3 | 17.7 | 14.3 |
| of Tie layer | | | | | | | | |
| gsm | | | | | | | | |
| Wt % of MA | 9.3 | 21.4 | 22.8 | 22.8 | 22.8 | 22.8 | 22.8 | 22.8 |
| in tie layer | | | | | | | | |
| PE Barrier | X | X | X | Y | Y | Y | Y | Y |
| Film layer | | | | | | | | |
| (65%) | | | | | | | | |
| Nonwoven | SB | SB | SB | SMS | SMS | SB | SB | SB |
| Fabric layer | | | | | | | | |
| type | | | | | | | | |

Off-Line Peel Testing of Seals Formed with Examples EX-1B, EX-5B, and C-EX.1-C-EX.6

Peel testing was performed in the MD and CD, using an INSTRON 5500R Model 1122 tester on samples cut to 2.5 cm (1 inch) wide and 10.2 cm (4 inch) long in the direction of peel testing, placed in a testing environment of 48° C. The seal sample specimens were created by impact sealing the coextruded Tie-layer+PE Barrier Film+Nonwoven constructions of Examples EX-1B, EX-5B, and C-EX.1-C-EX.6 to a 19 micrometer (0.75 mil) thick blown polyethylene film at a temperature of 155° C. (311° F.), 0.5 second dwell time and a pressure of 276 kPa (40 PSI) using a Packaging Industries Inc AS/2 series heat sealer with a 30.5 cm (12 inch) bar. The 19-micrometer (0.75-mil) thick blown polyethylene film was product PF5512, a thin film manufactured from thermoplastic polyethylene resins, available from AEP Industries Inc., of Mankato, Minn. In this impact sealing process, a piece of 12.7-micrometer (0.5-mil) thick PET film was used as a protective buffer layer between the heat sealer bar and the blown polyethylene film.

TABLE 8
Offline Seal Test

| | Machine Direction | | | | Cross Direction | | | |
|---|---|---|---|---|---|---|---|---|
| Example | Ave | Std | Max | Std | Ave | Std | Max | Std |
| Number | Force | Dev | Force | Dev | Force | Dev | Force | Dev |
| Control | 2.08 | 0.38 | 2.43 | 0.39 | 2.08 | 0.92 | 2.43 | 0.77 |
| Comp. C- | 1.87 | 0.34 | 2.23 | 0.22 | 1.91 | 0.96 | 2.49 | 0.67 |
| EX.6 | | | | | | | | |
| Comp. C- | 2.02 | 0.97 | 2.85 | 0.43 | 2.42 | 0.72 | 2.99 | 0.50 |
| EX.5 | | | | | | | | |
| Comp. C- | 1.18 | 0.36 | 2.56 | 0.13 | 2.04 | 0.63 | 3.30 | 0.38 |
| EX.4 | | | | | | | | |
| Comp. C- | 2.19 | 0.38 | 2.59 | 0.35 | 2.60 | 0.71 | 2.89 | 0.58 |
| EX.3 | | | | | | | | |
| Comp. C- | 1.95 | 0.50 | 2.61 | 0.34 | 2.70 | 0.70 | 3.20 | 0.47 |
| EX.2 | | | | | | | | |
| Comp. C- | 1.70 | 0.18 | 2.05 | 0.20 | 2.21 | 0.68 | 2.77 | 0.48 |
| EX.1 | | | | | | | | |
| EX-5B | 2.01 | 0.98 | 2.63 | 0.65 | 2.39 | 0.63 | 3.05 | 0.34 |
| EX-1B | 3.39 | 0.25 | 3.59 | 0.25 | 3.36 | 0.59 | 3.72 | 0.56 |

Control: Commercially available 3M BAIR HUGGER UPPER BODY BLANKET, Model 522

### Patient Warming Device Final Product—Pressure Seal Testing

Rolls of the (Tie-layer+PE Barrier Film+Nonwoven) Examples material prepared in Examples EX-1B, EX-5B, and C-EX.1 were converted into actual patient warming devices (final product) approximately equivalent to 3M BAIR HUGGER Upper Body Blanket, Model 522, see FIG. 5A, by attaching the PF5512 blown polyethylene film (19 micrometer (0.75 mil) thick) to the nonwoven side of the (nonwoven+tie-layer) laminate construction. Including providing air inlet ports 608 as shown in FIG. 5A. Prior to the sealing operation the coextruded (Tie-layer+PE Barrier Film+Nonwoven) material was perforated per the specific Model 522 product design. The perforations designed to function as warm air openings/nozzles for patient warming. The attachment between the coextruded (Tie-layer+PE Barrier Film+Nonwoven) material and the blown film was performed to create linear and transverse seals by passing the coextruded (Tie-layer+PE Barrier Film+Nonwoven) material and the blown LLDPE film through rotary compression sealers at 185° C. and an appropriate fixed gap. This resulted in forming inflatable tubes of the LLDPE blown polyethylene film on the film side of the coextruded (Tie-layer+PE Barrier Film+Nonwoven) material.

The patient warming devices thus manufactured were tested for product functionality by pumping hot air at 43-45° C., (measured at the blower/warming unit hose exit, of 3M BAIR HUGGER 500 series warming unit) into an air inlet port in the blown film tube assembly at an air pressure of 298.6 Pascal (1.2 inches of water column) A Seal Test result of “PASS” indicated the seals in the prepared Example patient warming device remained intact and functional for 10 minutes under the pressure and temperature described above. A Seal Test result of “FAIL” indicated the seals in the prepared Example patient warming device did not remain intact or functional for 10 minutes under the pressure and temperature described above. Table 9 shows the results of Pressure Seal Testing of replicate samples of the patient warming devices prepared to mimic the 3M BAIR HUGGER Upper Body Blanket, Model 522, using the (Tie-layer+PE Barrier Film+Nonwoven) constructions of EX-1B, EX-5B and C-EX.1.

TABLE 9
Pressure Seal Testing of Patient Warming Device Product

| Tie-layer + PE Barrier Film + Nonwoven | | |
|---|---|---|
| Example Material | n = 3 | n = 10 |
| Comparative Example C-EX. 1 | FAIL | Not tested |
| Example EX-5B | FAIL | Not Tested |
| Example EX-1B | PASS | PASS |

Peel Testing of Seals Excised from Patient Warming Device Products

Rectangular linear seal samples of 2.54 cm (1 inch) wide by 10.2 cm (4 inch) were excised (cut out) from the patient warming products (prepared to mimic 3M BAIR HUGGER Upper Body Blanket, Model 522) prepared above. The seal samples were peel tested for peak load along the longer side (CD) and along the short side (MD) at 5-cm (2-inch) gage length and 30.5 cm (12 inch) per minute pull rate. The peel testing of these seal samples was performed on an INSTRON 5500R Model 1122 tester equipped with a 48° C. heating chamber, an environment simulating the hot air temperature as in actual product use condition.

TABLE 10
Peel Testing of Seals Cut from Prepared Patient Warming Product

| | Machine Direction | | | | Cross Direction | | | |
|---|---|---|---|---|---|---|---|---|
| | Ave | Std | Max | Std | Ave | Std | Max | Std |
| Run | Force | Dev | Force | Dev | Force | Dev | Force | Dev |
| Control | 2.21 | 0.20 | 2.50 | 0.21 | 3.05 | 0.13 | 3.22 | 0.14 |
| C-EX.1 | 1.06 | 0.29 | 2.38 | 2.38 | 0.78 | 0.43 | 2.11 | 0.36 |
| EX-5B | 1.41 | 0.24 | 1.84 | 0.20 | 2.06 | 0.31 | 2.59 | 0.13 |
| EX-1B | 1.21 | 0.45 | 2.47 | 0.20 | 3.11 | 0.25 | 3.40 | 0.23 |

Control: Commercially available 3M BAIR HUGGER UPPER BODY BLANKET, Model 522

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. An inflatable medical article comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer to the polyolefin-containing layer;
wherein the tie layer comprises a copolymer prepared from monomers comprising at least one olefin monomer and up to 22 wt-% of at least one polar monomer, wherein the copolymer has a Vicat softening temperature of greater than 45° C.;
an optional sheet; and
at least one inflatable chamber formed between the polyolefin-containing layer and the tie layer, or between the polyester-containing layer and the tie layer, or between the polyolefin-containing layer and the optional sheet when such sheet is present.

2. The inflatable medical article of claim 1 further comprising a plurality of engineered openings for fluid communication between the at least one inflatable chamber and the environment.

3. The inflatable medical article of claim 1 wherein the at least one inflatable chamber is formed between the polyolefin-containing layer and the tie layer.

4. The inflatable medical article of claim 1 wherein the at least one inflatable chamber is formed between the polyester-containing layer and the tie layer.

5. An inflatable medical article comprising:
a laminate comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer to the polyolefin-containing layer;
wherein the tie layer comprises a copolymer prepared from monomers comprising at least one olefin monomer and up to 22 wt-% of at least one polar monomer, wherein the copolymer has a Vicat softening temperature of greater than 45° C.; and
a sheet bonded to the laminate to create at least one inflatable chamber.

6. The inflatable medical article of claim 5 wherein the polyolefin-containing layer is a film layer.

7. The inflatable medical article of claim 5 further comprising a plurality of engineered openings for fluid communication between the at least one inflatable chamber and the environment.

8. The inflatable medical article of claim 5 wherein the sheet is bonded to the polyolefin-containing layer of the laminate.

9. The inflatable medical article of claim 5 wherein the tie layer has a Vicat softening temperature of greater than 45° C.

10. The inflatable medical article of claim 5 wherein the polyester-containing layer is fluid repellent.

11. The inflatable medical article of claim 5 wherein the polyester-containing layer is a first polyester-containing layer, the polyolefin-containing layer has a first major surface and a second major surface, and the tie layer is a first tie layer bonding the first polyester-containing layer to the first major surface of the polyolefin-containing layer, and the laminate further comprises:
a second polyester-containing layer comprising a fabric layer comprising at least one nonwoven web of fibers comprising an aliphatic polyester, wherein at least a portion of the aliphatic polyester is exposed at the surface of the fibers; and
a second tie layer bonding the first polyester-containing layer to the second major surface of the polyolefin-containing layer;
wherein the second tie layer comprises a copolymer prepared from monomers comprising at least one olefin monomer and up to 22 wt-% of at least one polar monomer, wherein the copolymer has a Vicat softening temperature of greater than 45° C.

12. The inflatable medical article of claim 5 wherein the polyester-containing layer comprises two or more nonwoven webs of fibers comprising an aliphatic polyester, wherein at least a portion of the aliphatic polyester is exposed at the surface of the fibers.

13. The inflatable medical article of claim 5 wherein the laminate is a first laminate, and the sheet is in the form of a second laminate comprising:
a polyester-containing layer comprising a fabric layer comprising at least one nonwoven web of fibers comprising an aliphatic polyester, wherein at least a portion of the aliphatic polyester is exposed at the surface of the fibers;
a polyolefin-containing layer comprising a polyolefin film, a nonwoven web comprising polyolefin fibers, or a combination thereof; and
a tie layer bonding the polyester-containing layer of the second laminate to the polyolefin-containing layer of the second laminate;
wherein the tie layer comprises a copolymer prepared from monomers comprising at least one olefin monomer and up to 22 wt-% of at least one polar monomer, wherein the copolymer has a Vicat softening temperature of greater than 45° C.

14. (canceled)

15. The inflatable medical article of claim 5 which can withstand inflation at a pressure of 2.0 inches water (50 mm water) with air at a temperature greater than 38° C. without separation of the polyester-containing layer from the polyolefin-containing layer.

16. (canceled)

17. The inflatable medical article of claim 5 wherein at least 50% of the surface area of the exterior surface area of the fibers of the polyester-containing layer and/or at least 50% of the surface area of the nonwoven of the polyester-containing layer includes an aliphatic polyester.

18. (canceled)

19. The inflatable medical article of claim 5 wherein the tie layer comprises a copolymer prepared from monomers comprising at least one olefin monomer and at least 7 wt-% of at least one polar monomer.

20-23. (canceled)

24. The inflatable medical article of claim 5 wherein the tie layer comprises a copolymer prepared from monomers comprising at least one olefin monomer and up to 22 wt-% of at least one polar monomer, wherein the copolymer has a Vicat softening temperature of greater than 55° C.

25-28. (canceled)

29. The inflatable medical article of claim 5 which is in the form of a blanket, a pad, or a garment.

30. An inflatable medical article comprising:
a polyester-containing layer comprising a fabric layer comprising a nonwoven web of fibers comprising an aliphatic polyester, wherein at least a portion of the aliphatic polyester is exposed at the surface of the fibers;
a polyolefin-containing film layer; and
a tie layer bonding the polyester-containing layer to the polyolefin-containing layer;
wherein the tie layer comprises a copolymer having a Vicat Softening Temperature of at least 50° C., which is prepared from monomers comprising at least one olefin monomer and at least one polar monomer in an amount of 5-18 wt-%;
wherein the inflatable medical article can withstand inflation at a pressure of 2.0 inches water (50 mm water) with air at a temperature greater than 38° C. without separation of the fabric layer from the film layer; and
a sheet bonded to the polyolefin-containing film layer to create at least one inflatable chamber.

31-34. (canceled)

35. A patient warming device comprising the inflatable medical article of claim 1 and a convective apparatus integrated with or attached to the inflatable medical article.
